(19) United States
(12) Patent Application Publication
(10) Pub. No.: US 2025/0319428 A1
(43) Pub. Date: Oct. 16, 2025

(54) HIGH FLOW FILTER AND METHOD OF USE

(71) Applicant: MESOBLAST INTERNATIONAL SÀRL, Meyrin (CH)

(72) Inventors: Justin HORST, New York, NY (US); Suman ABRAHAM, New York, NY (US)

(21) Appl. No.: 18/869,064

(22) PCT Filed: May 26, 2023
(86) PCT No.: PCT/IB2023/055430

§ 371 Date: Nov. 25, 2024

Related U.S. Application Data
(60) Provisional application No. 63/365,393, filed on May 26, 2022.
(60) Provisional application No. 63/482,768, filed on Feb. 1, 2023.

(51) Int. Cl. B01D 29/56; B01D 29/05; C12M 1/00; C12M 1/26

(52) U.S. Cl. CPC B01D 29/56; B01D 29/05; C12M 23/14; C12M 29/04; C12M 33/14

(57) ABSTRACT

A filter is disclosed. The filter comprises: a flexible container defining an inlet port toward an inlet end of the container and an outlet port toward an outlet end of the container, the container having one or more walls connecting the inlet and outlet ends; and a first filter unit and a second filter unit spaced apart within the container, wherein: the first filter unit is coupled to the container to define an inlet chamber in fluid communication with the inlet port; the second filter unit is coupled to the container to define an outlet chamber in fluid communication with the outlet port; and the first and second filter units define an intermediate chamber between the inlet and outlet chambers, the first filter unit being configured to filter a fluid flowing from the inlet chamber to the intermediate chamber, and the second filter unit being configured to filter the fluid flowing from the intermediate chamber to the outlet chamber; wherein the inlet chamber comprises a first trough relative to a first peak; wherein the intermediate chamber comprises a second trough relative to a second peak; and wherein the outlet port and the second filter unit are configured to be spaced apart when the fluid is flowing through the second filter unit into the outlet chamber.

### PRIORITY APPLICATIONS

The present application claims priority to and benefit U.S. provisional application 63/365,393 filed May 26, 2022, and to U.S. provisional application 63/482,768 filed Feb. 1, 2023, each of which is herein incorporated by reference in its entirety for all purposes.

### TECHNICAL FIELD

The present disclosure relates to devices for preparing cell compositions with reduced particulates and/or cell aggregates, and methods of using said devices. Some embodiments of the present disclosure relate to filtration devices configured to maintain a relatively high flow rate of filtered cell composition exiting the filtration device.

### BACKGROUND

Several cell therapy products for regenerative or immune therapy applications have advanced to clinical evaluation and market authorization. The manufacturing process for such products typically involves culturing the cells in the presence of non-autologous serum and cell harvesting by trypsin digestion.

At several stages during the manufacturing process, the cells are exposed to extrinsic materials. At these stages, there is a risk that the cells may be contaminated with one or more particulates, such as cotton fibres, cellulose, salt crystals, rubber, plastics, glass etc. Such particulates are potentially harmful to the cells and/or to the recipient of the resulting cell therapy, if the particulates are incorporated into the final product. Filtering is generally required to remove particulates before a final composition is provided for therapy. However, filtering cellular compositions is not straightforward in view of one or more factors such as the complex nature of cell culture medium and, the tendency for cells to aggregate, in particular in the context of large scale cell culture. Clearly, there is an unmet need in the art for preparing cellular compositions, in particular in the context of therapeutic cellular compositions.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

Some embodiments relate to a filter comprising:
a flexible container defining an inlet port toward an inlet end of the container and an outlet port toward an outlet end of the container, the container having one or more walls connecting the inlet and outlet ends; and
a first filter unit and a second filter unit spaced apart within the container, wherein:
   the first filter unit is coupled to the container to define an inlet chamber in fluid communication with the inlet port;
   the second filter unit is coupled to the container to define an outlet chamber in fluid communication with the outlet port; and
   the first and second filter units define an intermediate chamber between the inlet and outlet chambers, the first filter unit being configured to filter a fluid flowing from the inlet chamber to the intermediate chamber, and the second filter unit being configured to filter the fluid flowing from the intermediate chamber to the outlet chamber;
wherein the inlet chamber comprises a first trough relative to a first peak;
wherein the intermediate chamber comprises a second trough relative to a second peak; and
wherein the outlet port and the second filter unit are configured to be spaced apart when the fluid is flowing through the second filter unit into the outlet chamber.

Some embodiments relate to a filter comprising:
a container defining an inlet port toward an inlet end of the container and an outlet port toward an outlet end of the container, the container having flexible front and back walls connecting the inlet and outlet ends; and
a first filter unit and a second filter unit spaced apart within the container, wherein:
   the first filter unit is coupled to the front and back walls of the container to define an inlet chamber in fluid communication with the inlet port;
   the second filter unit is coupled to the front and back walls of the container to define an outlet chamber in fluid communication with the outlet port; and
   the first and second filter units define an intermediate chamber between the inlet and outlet chambers, the first filter unit being configured to filter fluid flowing from the inlet chamber to the intermediate chamber, and the second filter unit being configured to filter fluid flowing from the intermediate chamber to the outlet chamber;
wherein the first filter unit is coupled to the front wall along a first front seam, and the second filter unit is coupled to the front wall along a second front seam;
wherein the first filter unit is coupled to the back wall at a first back seam, and the second filter unit is coupled to the back wall at a second back seam;
wherein a distance between the first and second front seams is less than a distance between the first and second back seams;
wherein the outlet port and the second filter unit are configured to be spaced apart when the fluid is flowing through the second filter unit into the outlet chamber.

The container may be a flexible bag. The walls may be flexible. The distance between the first and second front seams may be 1 inch or less. The second back seam may angle the second filter unit and the outlet port away from each other. The front wall may be configured to bulge away from the second filter unit.

The outlet port and the second filter unit may be configured to be spaced apart by a spacer system. The spacer system may be configured to space apart the outlet port and the second filter unit, and at least one of: (i) the front wall and the first filter unit; (ii) the back wall and the first filter unit; and (iii) the back wall and the second filter unit.

The spacer system may comprise a separator. The spacer system may comprise a clip. The spacer system may comprise a magnet system. The spacer system may comprise a brace.

The first filter unit may be connected to the front wall of the container at an acute angle to define an inlet chamber trough. The first filter unit may be connected to the back wall of the container at an obtuse angle. The first filter unit may comprise a mesh defining pores, the pores having an average pore size of 130 μm to 170 μm. The first filter unit pores may have an average pore size of 150 μm. The first filter unit may be substantially flat.

The second filter unit may be connected to the back wall of the container at an acute angle to define an intermediate chamber trough. The second filter unit may be connected to the front wall of the container at an acute angle. The second filter unit may comprise a mesh defining pores, the pores having an average pore size of 20 μm to 60 μm. The second filter unit pores may have an average pore size of 40 μm. The second filter unit may be substantially flat.

The inlet chamber may have a volume that is approximately 0.9 to 3.2 times the surface area of the first filter unit. The intermediate chamber may have: (i) a volume that is approximately 1.9 to 4.0 times the surface area of the first filter unit; or (ii) a volume that is approximately 2.7 to 5.6 times the surface area of the second filter unit. The outlet chamber may have a volume that is approximately 1.6 to 4.5 times the surface area of the second filter unit. The ratio of volumes of the inlet chamber to the intermediate chamber to the outlet chamber may be approximately (i) 1:1:1; or (ii) 1:2:1; or (iii) 1:2:2; or (iv) 2:2:1; or (v) 1:3:2.

At least one of the chambers may have a substantially triangular or substantially trapezoidal cross section, as viewed parallel to a direction of fluid flow from the inlet port to the outlet port.

Some embodiments relate to a filter comprising:
a flexible bag defining an inlet port toward an inlet end of the bag and an outlet port toward an outlet end of the bag, the bag having opposed front and back walls connecting the inlet and outlet ends;
a first filter unit and a second filter unit spaced apart within the bag, wherein:
   the first filter unit is coupled to the bag to define an inlet chamber in fluid communication with the inlet port;
   the second filter unit is coupled to the bag to define an outlet chamber in fluid communication with the outlet port;
   the first and second filter units define an intermediate chamber between the inlet and outlet chambers, the first filter unit being configured to filter fluid flowing from the inlet chamber to the intermediate chamber, and the second filter unit being configured to filter fluid flowing from the intermediate chamber to the outlet chamber; and
wherein the outlet port and the second filter unit are configured to be spaced apart when the fluid is flowing through the second filter unit into the outlet chamber.

The first filter unit and the second filter unit may not be parallel.

Some embodiments relate to a kit for filtering cells, the kit comprising:
the filter as described above;
an inlet conduit, the inlet conduit comprising:
   a first inlet tube and a second inlet tube, the first and second inlet tubes fluidly connected to a manifold;
   a filling tube, the filling tube in fluid communication with the manifold and adapted to receive fluid from at least one of the first and second inlet tubes; and
   an inlet coupling adapted to connect the filling tube to the inlet port of the filter and allow fluid communication with the inlet chamber;
an outlet conduit comprising:
a drain tube; and
an outlet coupling adapted to connect the drain tube to the outlet port of the filter and allow fluid communication with the outlet chamber and the drain tube.

The inlet tubes and the filling tube may form a Y-shape or a T-shape. The inlet tubes may have an inner diameter of ⅛ inches and an outlet diameter of ¼ inches. The filling tube may have an inner diameter of ⅛ inches and an outlet diameter of ¼ inches. The drain tube may have an inner diameter of ⅛ inches and an outlet diameter of ¼ inches. The kit may further comprise an outlet valve or clamp configured to control fluid flow through the drain tube.

One or more or all of the container, the bag, the filter units 120, 130, or the tubes may be made of DMSO (dimethyl sulfoxide) compatible plastic, preferably a plastic comprising one or more of polyethylene terephthalate (PET), High Density Polyethylene (HDPE), Low Density Polyethylene (LDPE), Polytetrafluoroethylene (PTFE), Poly Vinyl Chloride (PVC), Thermo Plastic Elastomer (TPE), and Polypropylene (PP).

Some embodiments relate to a filter comprising:
a container defining an inlet port toward an inlet end of the container and an outlet port toward an outlet end of the container, the container having one or more walls connecting the inlet and outlet ends; and
a first filter unit and a second filter unit spaced apart within the container, wherein:
   the first filter unit is coupled to the container to define an inlet chamber in fluid communication with the inlet port;
   the second filter unit is coupled to the container to define an outlet chamber in fluid communication with the outlet port; and
   the first and second filter units define an intermediate chamber between the inlet and outlet chambers, the first filter unit being configured to filter a fluid flowing from the inlet chamber to the intermediate chamber, and the second filter unit being configured to filter the fluid flowing from the intermediate chamber to the outlet chamber;
wherein the inlet chamber comprises a first trough relative to a first peak; and
wherein the intermediate chamber comprises a second trough relative to a second peak.

Some embodiments relate to a filter comprising:
a container defining an inlet port toward an inlet end of the container and an outlet port toward an outlet end of the container, the container having opposed front and back walls connecting the inlet and outlet ends; and
a first filter unit and a second filter unit spaced apart within the container, wherein:
   the first filter unit is coupled to the front and back walls of the container to define an inlet chamber in fluid communication with the inlet port;
   the second filter unit is coupled to the front and back walls of the container to define an outlet chamber in fluid communication with the outlet port; and
   the first and second filter units define an intermediate chamber between the inlet and outlet chambers, the first filter unit being configured to filter fluid flowing from the inlet chamber to the intermediate chamber, and the second filter unit being configured to filter fluid flowing from the intermediate chamber to the outlet chamber;
wherein the first filter unit is coupled to the front wall along a first front seam, and the second filter unit is coupled to the front wall along a second front seam;
wherein the first filter unit is coupled to the back wall at a first back seam, and the second filter unit is coupled to the back wall at a second back seam; and
wherein a distance between the first and second front seams is less than a distance between the first and second back seams.

Some embodiments relate to a filter comprising:
a flexible bag defining an inlet port toward an inlet end of the bag and an outlet port toward an outlet end of the bag, the bag having opposed front and back walls connecting the inlet and outlet ends;
a first filter unit and a second filter unit spaced apart within the bag, wherein:
   the first filter unit is coupled to the bag to define an inlet chamber in fluid communication with the inlet port;
   the second filter unit is coupled to the bag to define an outlet chamber in fluid communication with the outlet port; and
   the first and second filter units define an intermediate chamber between the inlet and outlet chambers, the first filter unit being configured to filter fluid flowing from the inlet chamber to the intermediate chamber, and the second filter unit being configured to filter fluid flowing from the intermediate chamber to the outlet chamber.

The filter or kit as described above may be used for filtering stem cell culture medium.

Some embodiments relate to a method of filtering a stem cell culture medium, the method comprising using the filter or the kit as described above.

Some embodiments relate to a method of purifying a cell composition, comprising passing cultured cells through a dual screen mesh filter, thereby reducing visible particulates and/or cell aggregates, wherein the dual screen mesh filter comprises a first filter screen with an average pore size of between 130 μm and 170 μm and a second filter screen with an average pore size of between 20 μm and 60 μm.

The cultured cells may be provided in serum free cell culture medium. The cells may be culture expanded. The cells may be mesenchymal lineage precursor or stem cells (MLPSC)s.

After passing the cells through the dual screen mesh filter, recovery of viable cell concentration is between (i) 60% and 100%; or (ii) 70% and 90%.

The purified cell composition may exhibit a D90 of less than 150 μm, preferably less than 100 μm, more preferably less than 50 μm. The purified cell composition may be substantially free of visible particles.

Some embodiments relate to a filter comprising:
a flexible container defining an inlet port toward an inlet end of the container and an outlet port toward an outlet end of the container, the container having one or more walls connecting the inlet and outlet ends;
a first filter unit comprising a first filter mesh; and
a second filter unit comprising a second filter mesh;
wherein the first filter unit and the second filter unit are spaced apart within the container;
wherein the first filter unit is coupled to the container to define an inlet chamber in fluid communication with the inlet port;
wherein the second filter unit is coupled to the container to define an outlet chamber in fluid communication with the outlet port; and
wherein the first and second filter units define an intermediate chamber between the inlet and outlet chambers, the first filter unit being configured to filter a fluid flowing from the inlet chamber to the intermediate chamber, and the second filter unit being configured to filter the fluid flowing from the intermediate chamber to the outlet chamber;
wherein the inlet chamber comprises a first trough relative to a first peak;
wherein the intermediate chamber comprises a second trough relative to a second peak; and
wherein at least one of the first filter mesh and the second filter mesh is configured to be spaced apart from the one or more walls when the fluid is flowing through the filter.

Some embodiments relate to a filter comprising:
a container defining an inlet port toward an inlet end of the container and an outlet port toward an outlet end of the container, the container having flexible front and back walls connecting the inlet and outlet ends;
a first filter unit comprising a first filter mesh; and
a second filter unit comprising a second filter mesh;
wherein the first filter unit and the second filter unit are spaced apart within the container, wherein:
   the first filter unit is coupled to the front and back walls of the container to define an inlet chamber in fluid communication with the inlet port;
   the second filter unit is coupled to the front and back walls of the container to define an outlet chamber in fluid communication with the outlet port; and
   the first and second filter units define an intermediate chamber between the inlet and outlet chambers, the first filter unit being configured to filter fluid flowing from the inlet chamber to the intermediate chamber, and the second filter unit being configured to filter fluid flowing from the intermediate chamber to the outlet chamber;
wherein the first filter unit is coupled to the front wall along a first front seam, and the second filter unit is coupled to the front wall along a second front seam;
wherein the first filter unit is coupled to the back wall at a first back seam, and the second filter unit is coupled to the back wall at a second back seam;
wherein a distance between the first and second front seams is less than a distance between the first and second back seams;
wherein the outlet port and the second filter unit are configured to be spaced apart when the fluid is flowing through the second filter unit into the outlet chamber; and
wherein at least one of the first filter mesh and the second filter mesh is configured to be spaced apart from the front or back walls when the fluid is flowing through the filter.

### BRIEF DESCRIPTION OF DRAWINGS

FIG. 1 is a perspective view of a filtration device, according to some embodiments;

FIGS. 2A-2D are side views of the filtration device of FIG. 1, in a filled configuration;

FIGS. 3A-3B are cross-section views of the filtration device of FIG. 1, as viewed along the line 3-3;

FIG. 4 is a perspective view of a kit comprising the filtration device of FIG. 1, according to some embodiments;

FIG. 5 shows an inlet conduit of a kit comprising the filtration device of FIG. 1, according to some embodiments; and

FIG. 6 shows an outlet conduit of a kit comprising the filtration device of FIG. 1, according to some embodiments.

### DETAILED DESCRIPTION

The present disclosure relates to devices for preparing cell compositions with reduced particulates and/or cell aggregates, and methods of using said devices.

In particular, some embodiments of the present disclosure relate to a filtration device which is suitable for filtering fluent materials, such as a cell composition or a cell culture medium or resuspension medium comprising the same. In an example, the cell composition comprises mesenchymal precursor lineage or stem cells. Some embodiments of the present disclosure relate to filtration devices configured to maintain a relatively high flow rate of filtered cell composition exiting the filtration device. The device may be referred to as a “dual screen mesh filter”. The dual screen mesh filter comprises a first filter unit and a second filter unit. In some embodiments, the filter units comprise a screen or sheet made from a mesh material (hence “dual screen mesh filter”). Dual screen mesh filters tend to be less prone to clogging when a viscous fluid is being filtered than other filters. Other embodiments of filter may include a pleated filter or depth-type filter.

In some embodiments, the filtration device may be sufficiently flexible so that they can be deformed to fit in tight or irregularly-shaped places. In certain applications, using a flexible filtration device may be particularly advantageous, in particular relative to more rigid structures, as the flexible filtration device can be easily positioned around more rigid components of an overarching cell culture and/or cell purification system comprising rigid structures such as stands, supports, pumps and the like. Filtration devices with this flexibility may comprise a flexible portion. In some embodiments, the filtration device is a flexible bag, similar to a saline bag.

The filter units are arranged such that during operation, fluid passing through the device flows through both the first and second filter units before exiting the device. The filter units may comprise a mesh having an average pore size which can be selected according to the size of the particles to be filtered from the fluent material. For example, a filter unit comprising a mesh having an average pore size of 150 μm, will in theory preclude particles which are >150 μm in diameter from flowing through the filter unit. Where the fluid flows through a series of filters, the pore size of successive filters may reduce in order to provide a gradual filtering. This may help reduce clogging of filters. For example, a first filter unit may have an average pore size of 150 μm, with a second filter unit having an average pore size of 40 μm.

The inventors have identified that in some circumstances, one or both of the mesh filters may sag or crease. The mesh filter(s) may sag or crease when a fluid flows through the mesh filter under gravity. In some circumstances, fluid may enter the filtration device faster than it passes through each of the filter units therein, thereby causing fluid to accumulate on the inlet side of the filter unit.

The mesh filter(s) may not sag or crease immediately, but only after an amount of fluid accumulates to cause the mesh filter(s) to sag under the weight of the accumulated fluid. Sagging or creasing of the mesh filter(s) may result in part of the mesh filter(s) to approach or contact a wall or inner surface of the filtration device, thereby impeding (by restricting or blocking) the flow of fluid through that part of the mesh filter(s). Impedance of the flow of fluid in this manner is more likely to occur in flexible or bag-like filtration devices, as the reduced rigidity (or lack thereof) in such filtration devices means that the filter(s) and the wall can be easily moved closer to each other.

The inventors have developed embodiments of devices and techniques to reduce or prevent the relative movement of the filter(s) and the walls of the filtration device.

The inventors have developed embodiments of devices and techniques which reduce or prevent the relative movement of the filter(s) and the walls of the filtration device while retaining the flexibility of the filtration device.

Advantageously, by providing the filtration device of the described embodiments, the filtration device (such as a flexible bag) may be impeded from or prevented from collapsing under gravity. In some embodiments, the filtration device of the described embodiments may hinder or prevent the filter(s) from resting against the wall of the filtration device (such as a flexible bag), thereby mitigating the restriction of fluid flow.

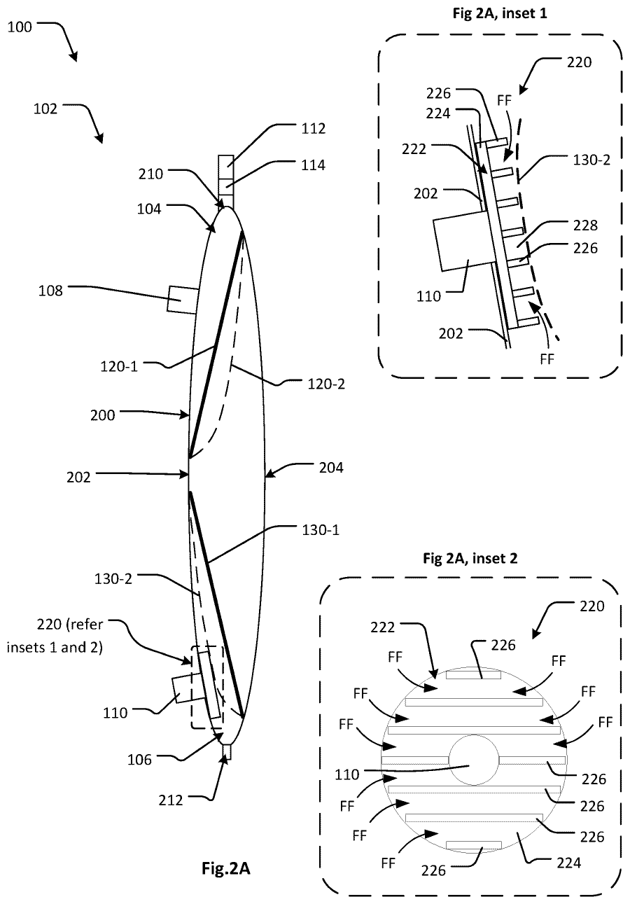

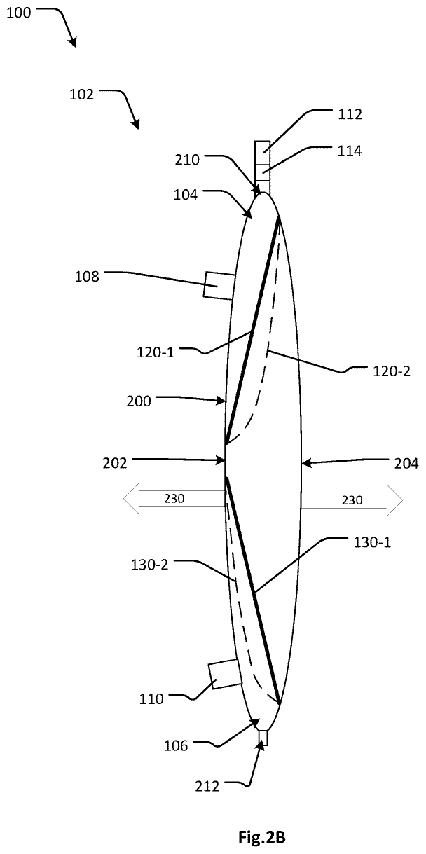

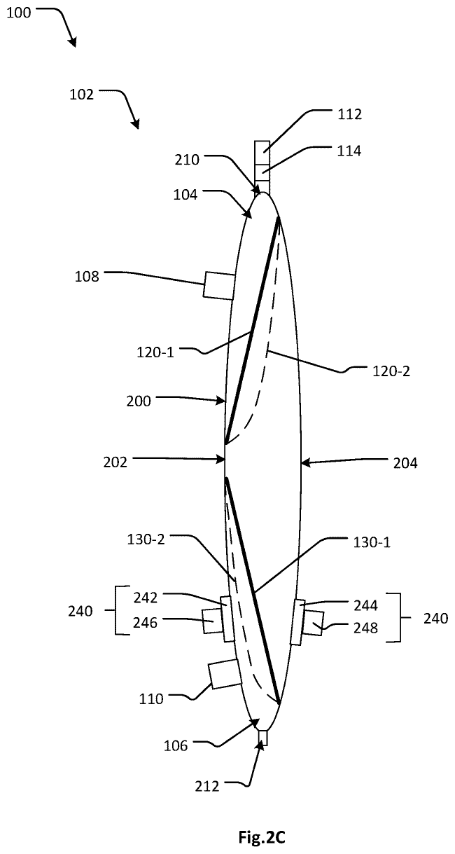

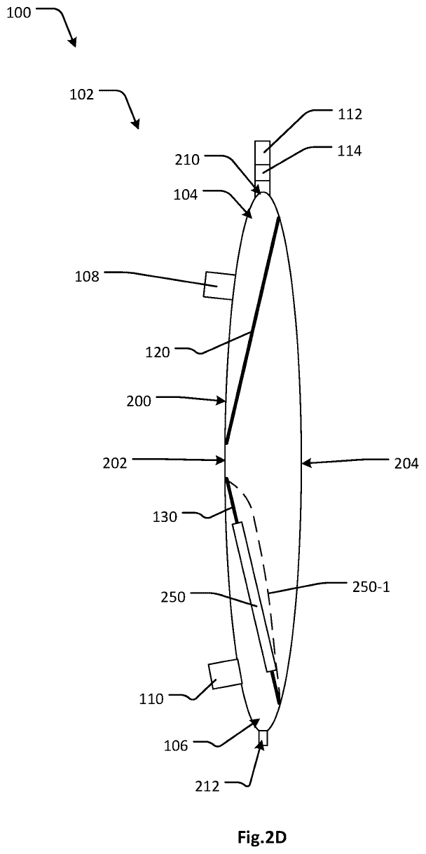

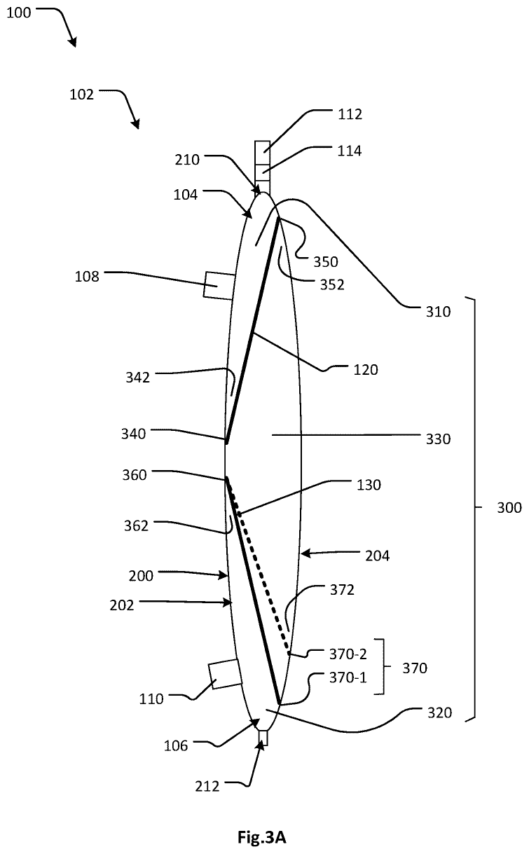

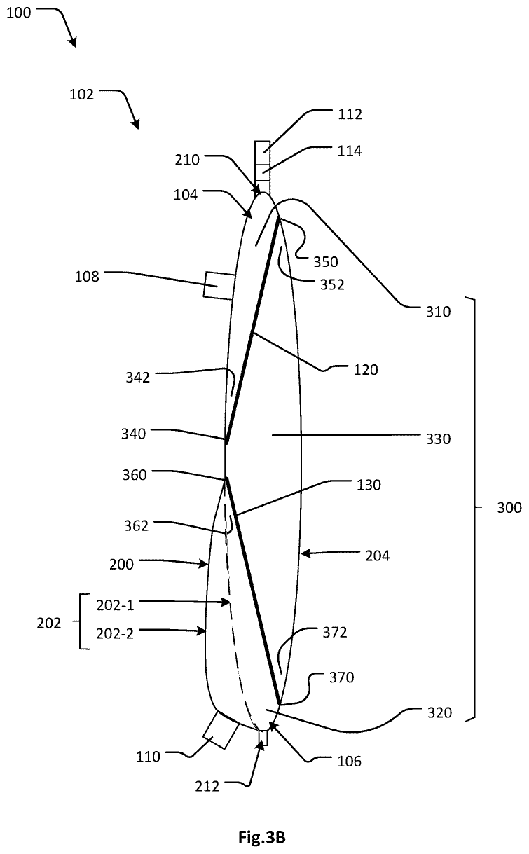

The disclosed embodiments may result in the flow of fluid through the filtration device being more consistent. The disclosed embodiments may result in the flow of fluid through the filtration device having a consistently higher flow rate compared to other filtration devices which do not use the disclosed embodiments. The disclosed embodiments are discussed in more detail below, with particular reference to FIGS. 2A-2D and FIGS. 3A and 3B.

### Filtration Device

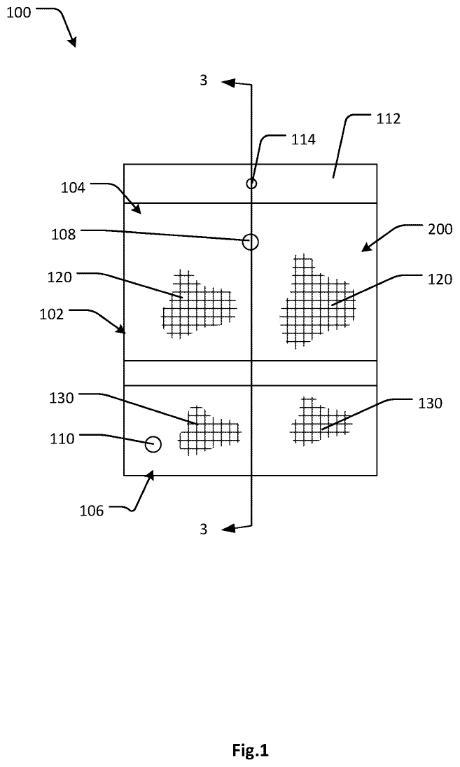

FIG. 1 shows an embodiment of a filtration device 100 for filtering a fluid, such as a cell composition. The filtration device 100 may alternatively be referred to as a filter. The filter 100 comprises a container 102, and a first filter unit 120 (partly shown in phantom) and a second filter unit 130 (partly shown in phantom) disposed in the container 102. The container 102 is adapted to receive the fluid, and the first filter unit 120 and the second filter unit 130 are configured to filter the fluid passing through the container 102. The filter units 120, 130 may each comprise a filter mesh having pores which are sized to permit matter of a specified size/type to flow through the filter units 120, 130. The filter mesh in the first filter unit 120 may have the same or different sized pores to the filter mesh in the second filter unit 130.

The pores are defined by pore-defining structures such as a body of the mesh. The mesh body and container 102 may be made from material that is compatible (e.g., resistant) to dimethyl sulfoxide (DMSO). Examples of DMSO compatible materials include DMSO compatible polymers. In some embodiments, the filter units 120, 130 are compliant with USP 788, which is a particulate matter test that quantifies the count and size of sub-visible particles in parenteral drugs. The USP 788 test involves using a light obscuration particle counter and counting particles on a filter by microscopy.

In some embodiments, the first filter unit 120 has an average pore size of between 130 μm and 170 μm, or between 140 and 160 μm. In some embodiments, the first filter unit 120 has an average pore size of about 150 μm.

In some embodiments, the second filter unit 130 has an average pore size of between 20 μm and 60 μm, or between 30 and 50 μm. In some embodiments, the second filter unit 130 has an average pore size of about 40 μm.

In some embodiments, the container 102 is a hollow body. The body may have a regular shape such as being substantially square (cubed) or rectangular (cuboid), like a carton. The container 102 may be sufficiently rigid to support its own weight, or sufficiently flexible so that it can be positioned in tight or irregularly-shaped places. The container 102 may comprise a combination of rigid and flexible portions. In some embodiments, the container 102 is a flexible bag, similar to a saline bag.

The container 102 has an inlet end 104 and an outlet end 106. The container 102 defines an inlet port 108 toward the inlet end 104, and defines an outlet port 110 toward the outlet end 106. In some embodiments, the inlet port 108 and the outlet port 110 are disposed generally opposite each other so that when the filtration device 100 is in use, the fluid may flow from the inlet port 108 toward the outlet port 110 by gravity. In some embodiments, the container 102 comprises a flange 112. The flange 112 may be disposed at the inlet end 104. The flange 112 may define an aperture 114 or connector through which the container 102 can be suspended from a hook (not shown). The aperture 114 allows the container 102 to be suspended or supported in an upright position so that the fluid may flow from the inlet port 108 toward the outlet port 110 by gravity while the filtration device 100 is in use.

FIGS. 2A-2D are side views of the filtration device 100. The container 102 comprises one or more walls 200 connecting the inlet and outlet ends 104, 106. The one or more walls 200 may comprise a front wall 202 and a back wall 204. The one or more walls 200 may further comprise side walls, side portions, or lateral ends (not shown) which connect the front and back walls 202, 204 to each other to define the main body of the container 102. In some embodiments, the front wall 202 and the back wall 204 are opposed (oppositely disposed). Some embodiments of the container 102 may further comprise a top connecting portion 210 and a bottom connecting portion 212. The top connecting portion 210 and bottom connecting portion 212 may be a seam that connects the front wall 202 and the back wall 204. The seam 210 and the flange 112 may be connected to each other; for example, the flange 112 may extend from the seam 210. In some embodiments, the inlet end 104, outlet end 106, and lateral ends comprise at least one seam or sterile welded/bonded edge which connects the one or more walls 200.

The first filter unit 120 and the second filter unit 130 are spaced apart within the container 102 and are connected to the walls 200 of the container 102. By spacing apart the first filter unit 120 and the second filter unit 130, more of the fluid can pass through the first filter unit 120 before passing through the second filter unit 130. The spaced apart filter units 120, 130 may reduce the likelihood of filter blockage, for example caused by the first filter unit 120 and the second filter unit 130 touching in a manner where the pores of one filter unit are blocked by the pore-defining structures of the other filter unit. The filter units 120, 130 may also potentially become stuck together if the fluid being filtered is sticky or particularly viscous.

When the filtration device 100 is suspended to allow fluid to flow through the filtration device 100 by gravity, the first filter unit 120 and the second filter unit 130 may be pulled relatively taut so that they are generally not sagging towards either of the walls 200. The tautened configuration of the first filter unit 120 and the second filter unit 130 is labelled as 120-1 and 130-1 respectively.

As discussed above, when the fluid is flowing through the first filter unit 120 toward the outlet port 110, the weight of the fluid on the first filter unit 120 may cause at least part of the first filter unit 120 to sag and move away from the inlet end 104. This sagged configuration of the first filter unit 120 is labelled as 120-2. In the sagged configuration 120-2, at least part of the first filter unit 120 may approach or contact a wall 200 of the container 102, thereby impeding or blocking flow of fluid through that part of the first filter unit 120. For example, in the sagged configuration 120-2, at least part of the first filter unit 120 may touch the back wall 204. This may reduce the surface area of the first filter unit 120 that is available to filter the fluid.

Similarly, when the fluid is flowing through the second filter unit 130 toward the outlet port 110, the weight of the fluid on the second filter unit 130 may cause at least part of the second filter unit 130 to sag and move away from the inlet end 104. This sagged configuration of the second filter unit 130 is labelled as 130-2. In the sagged configuration 130-2, at least part of the second filter unit 130 may approach or contact a wall 200 of the container 102, thereby impeding or blocking flow of fluid through that part of the second filter unit 130. For example, in the sagged configuration 130-2, at least part of the second filter unit 130 may touch the front wall 202. In some circumstances, at least part of the second filter unit 130 may touch the outlet port 110. This may reduce the surface area of the second filter unit 130 that is available to filter the fluid.

The abovementioned reduction in filtering surface area of the first filter unit 120 and the second filter unit 130 may substantially affect the rate of filtered fluid through the outlet port 110.

The inventors have identified that, in some embodiments, the rate of filtered fluid through the outlet port 110 may be affected more by the movement of the second filter unit 130 towards the wall 202, than by the movement of the first filter unit 120 towards the wall 204. The inventors consider that this may be because the second filter unit 130 has, on average, a smaller pore size than the first filter unit 120.

The inventors consider that where the filtration device 100 is or resembles a saline bag, the geometry of the bag may affect the flow of the fluid through the second filter unit 130. This is illustrated in FIG. 2A, for example, which shows a container 102 that is narrower at ends 104, 106 compared to the midsection therebetween. Accordingly, when the second filter 130 is in the sagged configuration 130-2, the second filter unit 130 has less distance to travel to contact the wall 202 of the container 102. In comparison, as the container 102 in FIG. 2A is wider in its midsection, when the first filter unit 120 is in its sagged configuration 120-2 it has a greater distance to travel to contact the wall 204 of the container 102.

In some embodiments, the outlet port 110 and the second filter unit 130 are configured to be spaced apart from each other. The outlet port 110 and the second filter unit 130 may be configured to be spaced apart from each other when the fluid is flowing through the second filter unit into the outlet chamber. In embodiments where the container 102 is flexible, like a saline bag, the distance between the outlet port 110 and the second filter unit 130 may vary so that in some configurations, the outlet port 110 and the second filter unit 130 are in contact, while in other configurations, the outlet port 110 and the second filter unit 130 are spaced apart.

With reference to FIGS. 2A-2D, the filtration device 100 may comprise a spacer system 220. The spacer system 220 is configured to space apart the outlet port 110 and the second filter unit 130. The spacer system 220 may enable the container 102 to move between a collapsed state and an expanded state, so that the outlet port 110 and the second filter unit 130 can be brought into contact and moved apart.

By spacing apart the second filter unit 130 from the front wall 202, the spacer system 220 enables the pores of the second filter unit 130 to remain unblocked or relatively clear, and allow fluid to continue passing through the second filter unit 130 and enter the outlet chamber 320.

The spacer system 220 may enable the flow rate of fluid through the second filter unit 130 to be consistent (or minimally reduced) regardless of whether the second filter unit 130 is in the tautened configuration 130-1 or the sagged configuration 130-2.

FIG. 2A shows an embodiment of the spacer system 220, in the form of a separator 222. The separator 222 comprises a separator body 224. The separator 222 is configured to be connected to the outlet port 110 on the inside of the container 102. The separator 222 and the outlet port 110 may thereby sandwich the front wall 202.

When the second filter unit 130 is in the sagged configuration 130-2, the separator 222 reduces the movement of the second filter unit 130 towards the outlet port 110. The separator 222 may limit the surface area of the second filter unit 130 that is able to come into contact with the front wall 202. FIG. 2A includes two inset views, labelled inset 1 and inset 2, which respectively show an embodiment of the separator 222. Inset 1 is a side view showing how the separator 222, according to some embodiments, may limit the surface area of the second filter unit 130 that is able to come into contact with the front wall 202. Inset 2 is an end view (viewed from the filter side) of the separator 222, according to some embodiments.

The separator body 224 may be disc shaped, with a thickness that sets the distance by which the second filter unit 130 is separated from the front wall 202. The separator 222 may be rigid. The separator 222 may be made from a similar material as the outlet port 110. The separator body 224 defines a plurality of apertures through which fluid can flow to the outlet port 110.

The separator 222 may further comprise at least one ridge or fence 226 extending from the separator body 224 to effectively increase the thickness of the separator 222 and increase the separation of the second filter unit 130 from the front wall 202. In some embodiments, the separator 222 comprises at least two of the ridges or fences 226. The fences 226 and the separator body 224 may define apertures or channels 228 in between adjacent fences 226. The channels 228 are configured to allow fluid to flow through the channels, as indicated by arrows FF on FIG. 2A (refer insets 1 and 2). The fences 226 may be configured to direct fluid flowing through the channels 228 towards the outlet port 110. In some embodiments, some of the fences 226 may define apertures or passages to allow fluid to flow between adjacent channels 228, towards the outlet port 110.

The fences 226 may be configured to allow the outlet port 110 to be unobstructed by the second filter unit 130. When the second filter unit 130 is in the sagged configuration 130-2, the second filter unit 130 is configured to abut the fences 226. The fences 226 are configured to support the second filter unit 130, spacing apart the second filter unit 130 and the separator body 224. Adjacent fences 226 may be spaced apart from each other so that the second filter unit 130 does not substantially sag between the fences 226 to block the channel 228. Outlet port 110 may then be unobstructed by the second filter unit 130. Fluid may then flow through the channels 228, as indicated by arrows FF on FIG. 2A (refer insets 1 and 2).

In some embodiments, the fences 226 are configured to direct fluid flowing through the channels 228 towards the outlet port 110. For example, the fences 226 may extend radially from the outlet port 110, which may be located in the central region of the separator body 224. In this configuration, all of the channels 228 may allow direct fluid flow towards the outlet port 110.

FIG. 2B shows an embodiment of the spacer system 220, in the form of a clip 230. The clip 230 is configured to be connected to the container 102 to hold apart the outlet port 110 and the second filter 130. In some embodiments, the clip 230 pinches the front wall 202 and the back wall 204. As the second filter 130 is connected to the front wall 202 and the back wall 204, holding apart the walls 202, 204 applies a tensioning force to the second filter 130 and keeps it separate from the outlet port 110.

FIG. 2C shows embodiment of the spacer system 220, in the form of a magnet system 240. The magnet system 240 comprises a first magnetic portion 242 that is coupled to the front wall 202, and a second magnetic portion 244 that is coupled to the back wall 204. The magnetic portions 242, 244 are configured to be connected to the respective walls 202, 204. This may be by adhesive, or storage in a pouch created by bonding/welding a layer of material onto the walls 202, 204, for example. The magnetic portions 242, 244 are separated from the fluid in the container 102 by the walls 202, 204, so that the fluid in the container 102 does not come into contact with the magnetic portions 242, 244 (for example, to avoid contamination of the cell composition). The magnetic portions 242, 244 are preferably located near to the outlet port 110.

In some embodiments, each of the magnetic portions 242, 244 is a magnet, with their respective polarities arranged so that magnets 242, 244 repel each other to keep the front and back walls 202, 204 apart. When the filtration device 100 is in use, the magnets 242, 244 may be magnetically attracted to adjacent metallic structure 246, 248. The adjacent metallic structure 246, 248 may be part of an exoskeleton (not shown) configured to support the filtration device 100. When the magnets 242, 244 are engaged with the adjacent metallic structure 246, 248, the magnet system 240 holds the front and back walls 202, 204 apart.

FIG. 2D shows an embodiment of the spacer system 220, in the form of a brace 250. The brace 250 is configured to be applied to the second filter unit 130 to support the mesh. The brace 250 may comprise a plurality of rigid members which are arranged to reduce the sagging of the mesh of the second filter unit 130. The inventors have identified that certain arrangements of the members may impede the flow of fluid through the second filter unit 130. The rigid members of the brace 250 may be arranged primarily at or towards the perimeter of the second filter unit 130. The rigid members of the brace 250 may be thin wires.

In some embodiments, the brace 250 comprises a convex portion, so that the second filter unit 130 is formed to be at least partially convex in shape, such as shown by the dashed line 250-1. The convexity of the second filter unit 130 arches the second filter unit 130 away from the outlet port 110. This means that when fluid accumulates on the second filter unit 130, the weight of the fluid first has to straighten the second filter unit 130, and is less likely to subsequently cause sagging (concavity) of the second filter unit 130.

Embodiments of the spacer system 220 may be combined to operate in combination. For example, the separator 222 may be used in conjunction with the magnet system 240. The spacer system 220 may be used to space apart any of the walls 200 and the filter units 120, 130, particularly in embodiments where the container 102 is flexible. The spacer system 220 may be configured to space apart the front wall 202 and the first filter unit 120. The spacer system 220 may be configured to space apart the front wall 202 and the second filter unit 130. The spacer system 220 may be configured to space apart the back wall 204 and the first filter unit 120. The spacer system 220 may be configured to space apart the back wall 204 and the second filter unit 130.

The aforementioned embodiments of the spacer system 220 are described and shown in FIGS. 2A-2D as being applied only to the second filter unit 130. However, the spacer system 220 can be similarly applied to the first filter unit 120. For example, the separator 222 could be placed on the back wall 204 to separate it from the first filter unit 120 when the first filter unit 120 is in the sagged configuration 120-2. In some embodiments, the spacer system 220 is applied to the first filter unit 120 instead of, or in addition to, the spacer system 220 being applied to the second filter unit 130.

As best shown in FIGS. 3A-3B, the first filter unit 120 and the second filter unit 130 are connected to the container 102 and separate the inside of the container 102 into a plurality of distinct regions or chambers 300. The chambers 300 are configured to be in fluid communication with each other through the first and second filter units 120, 130. The container 102 may comprise an inlet chamber 310 and an outlet chamber 320, with an intermediate chamber 330 disposed between the inlet chamber 310 and the outlet chamber 320. The spaced-apart disposition of the first and second filter units 120, 130 define the intermediate chamber 330 between the inlet and outlet chambers 310, 320. The first filter unit 120 may be configured to filter fluid flowing from the inlet chamber 310 to the intermediate chamber 330, and the second filter unit 130 may be configured to filter fluid flowing from the intermediate chamber 330 to the outlet chamber 320.

In some embodiments, the first filter unit 120 is coupled to the container 102 to define the inlet chamber 310. The inlet chamber 310 may span from the inlet end 104 to the first filter unit 120, which is coupled to the container 102; for example, coupled to at least the front and back walls 202, 204. The first filter unit 120 may be coupled to the container 102 (such as at the front wall 202) along a first front seam 340, and may be coupled to the container 102 (such as at the back wall 204) at a first back seam 350. The first front seam 340 and the first back seam 350 may extend to meet each other at the lateral ends 206, 208, such as in embodiments where the container 102 is similar in configuration to a saline bag.

The inlet chamber 310 is configured to be in fluid communication with the inlet port 108 so that fluid flowing into the inlet port 108 collects in the inlet chamber 310 and passes through the first filter unit 120. The first filter unit 120 is configured to filter a first matter (e.g. particulates) contained in the fluid. For example, the first matter may be particulates having a size larger than a pore size of the first filter unit 120. The first filter unit 120 may be configured to filter the first matter from the fluid flowing from the inlet chamber 310 to the intermediate chamber 330. The first matter remains on the surface of the first filter unit 120 while the rest of the fluid passes through.

If the first matter is allowed to accumulate on the surface of the first filter unit 120, more and more of the pores of the first filter unit 120 will be obstructed, thereby reducing the filtering efficiency. Accordingly, in some embodiments the inlet chamber 310 may comprise a first trough 342. In some embodiments, the first trough 342 is a recessed part of the inlet chamber 310. The first trough 342 is disposed relative to a first peak 352. In some embodiments, the first peak 352 is a projecting, optionally pointed, part of the inlet chamber 310. In some embodiments, the first peak 352 is defined by the first filter unit 120. The first trough 342 may also be defined as being relative to a first peak 352 of the adjacent intermediate chamber 330 (that lies on the other side of the first filter unit 120). In use, the first trough 342 is a lowermost portion of the inlet chamber 310 and the first peak 352 is a portion of the adjacent intermediate chamber 330 that is disposed higher than the first trough 342 (i.e., closer to the inlet end 104). In some embodiments, the inlet chamber 310 may be substantially wedge shaped. For example, the inlet chamber 310 may taper from at or near the inlet end 104 to the first trough 342.

The first filter unit 120 may be connected to at least one of the container walls 200 at an angle to define the first trough 342, and connected to another one of the container walls 200 at an angle to define the first peak 352. For example, the first trough 342 may be defined by the first filter unit 120 and the front wall 202. The angle defining the first trough 342 (as measured in the inlet chamber 310) may be an acute angle. The first trough 342 may be referred to as an inlet chamber trough. The first peak 352 may be defined by the first filter unit 120 and the back wall 204. The angle defining the first peak 352 (as measured in the intermediate chamber 330) may be an obtuse angle. When such an embodiment is in use, the first filter unit 120 would be tilted inside the container 102.

In use, the first filter unit 120 may be configured to direct the first matter into the first trough 342. The tilt of the first filter unit 120 means that particles that do not pass through the filter unit 120 may move along the filter mesh towards and into the trough 342 of the inlet chamber 310. For example, as the trough 342 is at the lowest point of the inlet chamber 310, gravity may encourage these particles to settle in the trough 342 instead of on the filter surface. In some embodiments, the fluid in the inlet chamber 310 may wash the first matter along the surface of the first filter unit 120 into the first trough 342.

Directing the first matter to accumulate in the first trough 342 may keep a larger proportion of the filter unit surface clear/unblocked, improving filtering efficiency and/or reducing the amount of time until the filter unit 120 (or filtration device 100) needs to be cleaned or replaced. By comparison, if a horizontally-arranged filter unit were used, these “rejected” particles (the first matter) would settle and accumulate against the pores of the horizontally-arranged filter unit and obstruct the flow of fluid therethrough. Accordingly, by directing “rejected” particles into the trough 342, the tilted filter unit 120 reduces the risk of pore blockage and allows a greater amount of fluid to pass through the filter unit 120.

The fluid is configured to pass through the first filter unit 120 and into the next chamber, which in some embodiments is the intermediate chamber 330. The top of the intermediate chamber 330 is defined by the first filter unit 120. In some embodiments, the second filter unit 130 is coupled to the container 102 (and spaced away from the first filter unit 120) to define the bottom of the intermediate chamber 330. In some embodiments, when the second filter unit 130 is coupled to the container 102, one side of the second filter unit 130 defines the bottom of the intermediate chamber 330 while the opposite side of the second filter unit 130 defines the outlet chamber 320.

In some embodiments, the intermediate chamber 330 comprises a second trough 372. In some embodiments, the second trough 372 is a recessed portion of the intermediate chamber 330. The second trough 372 is disposed relative to a second peak 362. In some embodiments, the second peak 362 is a projecting, optionally pointed, part of the intermediate chamber 330. In some embodiments, the second peak 362 is defined by the second filter unit 130. The second trough 372 may also be defined as being relative to a second peak 362 of the adjacent outlet chamber 320 (that lies on the other side of the second filter unit 130). In use, the second trough 372 is a lowermost portion of the intermediate chamber 330 (i.e., closer to the outlet end 106) and the second peak 362 is a portion of the adjacent outlet chamber 320 that is disposed higher than the second trough 372 (i.e., closer to the inlet end 104).

The second filter unit 130 may be connected to at least one of the container walls 200 at an angle to define the second trough 372, and connected to another one of the container walls 200 at an angle to define the second peak 362. For example, the second trough 372 may be defined by the second filter unit 130 and the back wall 204. The angle defining the second trough 372 (as measured in the intermediate chamber 330) may be an acute angle. The second trough 372 may be referred to as an intermediate chamber trough. The second peak 362 may be defined by the second filter unit 130 and the front wall 202. The angle defining the second peak 362 (as measured in the intermediate chamber 330) may be an acute angle. When such an embodiment is in use, the second filter unit 130 would be tilted inside the container 102.

The second filter unit 130 may be configured to filter a second matter from the fluid flowing from the intermediate chamber 330 to the outlet chamber 320. The second matter may be particulates of a different size or type to the first matter. The second filter unit 130 may cooperate with the first filter unit 120 to progressively filter matter from the fluid passing through the container 102. The progressive filtration of matter may improve yield of the desired fluid product, and may reduce the likelihood of the filter units 120, 130 becoming clogged due to attempting to remove too many particulates at the same time.

In some embodiments, the second matter may be particulates of the same size or type as the first matter, wherein the second filter unit 130 is configured to catch or trap any particulates that were not meant to pass through the first filter unit 120. Similar to the first filter unit 120 and the first trough 342, trapped particulates that accumulate on the second filter unit 130 may be directed into the second trough 372. The second trough 372 is configured to receive the second matter. For example, the fluid in the intermediate chamber 330 may wash the second matter along the surface of the second filter unit 130 into the second trough 372. Directing the second matter to accumulate in the second trough 372 may keep a larger proportion of the filter unit surface clear/unblocked, improving filtering efficiency and/or reducing the amount of time until the filter unit 130 (or filtration device 100) needs to be cleaned or replaced.

Fluid passing through the second filter unit 130 enters the outlet chamber 320. The outlet chamber 320 is in fluid communication with the outlet port 110, which allows the fluid in the outlet chamber 320 to drain out of the filter container 102. The fluid in the outlet chamber 320 contains the yield; that is, the desired fluid product that has been substantially isolated through removal of the particulates by the filter units 120, 130. In some embodiments, some trace amounts of particulates may have managed to pass through the filter units 120, 130, but the use of the filtration device 100 allows the amount of particulates in the filtered fluid to be substantially reduced compared to the unfiltered fluid. Further filtration may be used to reduce the amount of these remaining particulates.

The outlet chamber 320 may span from the outlet end 106 to the second filter unit 130, which is coupled to the container 102; for example, coupled to at least the front and back walls 202, 204. The second filter unit 130 may be coupled to the container 102 (such as at the front wall 202) along a second front seam 360, and may be coupled to the container 102 (such as at the back wall 204) at a second back seam 370. The second front seam 360 and the second back seam 370 may extend to meet each other at the lateral ends 206, 208, such as in embodiments where the container 102 is similar in configuration to a saline bag. In some embodiments, the outlet chamber 320 may be substantially wedge shaped. For example, the outlet chamber 320 may taper from at or near the outlet end 106 to the second peak 362.

The geometry of the container 102 may be altered to space apart the second filter unit 130 and the front wall 202/the outlet port 110, as similarly achieved by the spacer system 220. The spacer system 220 may generally be categorised as involving structural devices which mechanically separate the second filter unit 130 and the front wall 202. In comparison, the geometry of the container 102 does not rely on structural devices. However, the spacer system 220 can be used in conjunction with the altered geometry of the container 102.

FIGS. 3A and 3B show embodiments of the container 102 with an altered geometry.

The location of the second back seam 370 may be adjusted to further space apart the second filter unit 130 from the outlet port 110. Two example locations for the second back seam 370 are shown in FIG. 3A. The first location 370-1 is closer to the outlet end 106 compared to the second location 370-2. Accordingly, at the first location 370-1, the second filter unit 130 is closer to the outlet port 110 compared to the second location 370-2. When the second back seam 370 is at the second location 370-2, the indicative position of the second filter unit 130 is shown in dashed lines. The second location 370-2 may be chosen so that when in use, the outlet port 110 and the second filter unit 130 are further angled away from each other, as compared to the first location 370-1.

FIG. 3B shows an embodiment of the container 102 wherein the front wall 202 is configured to bulge away from the second filter unit 130. FIG. 3B shows the front wall 202 in a bulged configuration 202-2. For comparison, the front wall 202 is shown in broken lines in its “regular” configuration 202-1 (such as shown in FIG. 3A). In the bulged configuration 202-2, the front wall 202 may bulge between the second front seam 360 and the second back seam 370. The front wall 202 may bulge between the second front seam 360 and the bottom seam 212. The amount of material between the second front seam 360 and the second back seam 370 may be greater than in the embodiment shown in FIG. 3A (“regular” configuration 202-1), for example. This allows the front wall 202 of the container 102 to bulge as per the bulged configuration 202-2.

The outlet port 110 is coupled to the front wall 202. The outlet port 110 has a mass that is sufficient to deform the front wall 202. For example, when the filtration device 100 is arranged so that the fluid passes through the second filter unit 130 under gravity, gravity pulls the outlet port 110 downward, which in turn pulls the front wall 202 down and causes it to assume the bulged configuration 202-2. The outlet port 110 may be positioned closer to the lower end 106 to weigh down/pull the front wall 202 in the desired direction. When fluid passes through the second filter unit 130 and enters the outlet chamber 320, the weight of the fluid may cause the front wall 202 down to assume the bulged configuration 202-2.

In the bulged configuration 202-2, the front wall 202 (and/or outlet port 110) is moved away from the second filter unit 130. Even when the fluid accumulated on the second filter unit 130 causes the second filter unit 130 to sag, the front wall 202 (and/or outlet port 110) is still spaced away from the second filter unit 130, thereby leaving the pores of the second filter unit 130 unblocked or substantially clear and allowing fluid to continue passing through the second filter unit 130 and enter the outlet chamber 320.

Additional outlet ports 110 may be added to provide further locations for drainage of the fluid in the outlet chamber 320. In some embodiments, the outlet port 110 may be positioned generally perpendicular to the sagging of the filter unit 130, so that in the sagged configuration the filter unit 130 leaves the outlet port 110 substantially unobstructed.

As the first filter unit 120 and the second filter unit 130 are connected to the container 102 in a spaced apart arrangement, there is a front seam distance between the first and second front seams 340, 360, and a back seam distance between the first and second back seams 350, 370. The front seam distance and the back seam distance may be equal or unequal. In some embodiments, the front seam distance and/or the back seam distance is in the range of 0.5 inches to 5 inches. In some embodiments, the front seam distance and/or the back seam distance is approximately 1 inch.

In some embodiments, the filter units 120, 130 are substantially planar (flat), and so where the front seam distance and the back seam distance are equal, the filter units 120, 130 are parallel. This may result in the intermediate chamber 330 having a square or parallelogram cross section.

In some embodiments, the filter units 120, 130 are substantially planar (flat), and so where the front seam distance and the back seam distance are unequal, the filter units 120, 130 are not parallel to each other (such as shown in FIGS. 2A-2D and FIGS. 3A and 3B). When the filter units 120, 130 are not parallel to each other, this may reduce the likelihood of the filter units 120, 130 touching and blocking each other along a significant portion of their surface area. For example, if the filter is a flexible bag that is bent into a curved shape, the filter units 120, 130 may be caused to touch only at a specific point (despite the spacing or the non-parallel configuration) rather than across most of their surface area.

The filter units 120, 130 may be angled or tilted relative to each other in generally a “V” configuration, such as shown in FIGS. 2A-2D and FIGS. 3A and 3B. The filter units 120, 130 may be angled or tilted relative to each other so that the intermediate chamber 330 may generally resemble a triangle, trapezoid, or trapezium in cross section. In some embodiments, a minimum front seam distance is less than a minimum back seam distance.

The tilt of the filter units 120, 130 means that the first and second filter unit 120, 130 are angled relative to each other i.e. are not parallel (such as shown in FIGS. 2A-2D and FIGS. 3A and 3B). Combined with the spacing between the filter units 120, 130, this angle reduces the likelihood of the filter units 120, 130 contacting (and sticking to) each other across the majority of their filter surfaces, particularly in embodiments where the container 102 is flexible. This may reduce clogging and/or damage of the filter mesh. The inventors have found that if the filter meshes were positioned back-to-back, this may create depth and clog and withhold some cells, thus altering concentration and impacting yield. In this context, “depth” refers to the effective thickness of the filter when caused by the first filter unit 120 and the second filter unit 130 touching or being so close together such that the pores of one filter unit are blocked by the pore-defining structures of the other filter unit. For example, if the first filter unit 120 has a larger pore size than the second filter unit 130, placing the filter units 120, 130 close together can cause the first filter unit 120 to effectively have the same pore size as the second filter unit 130. Accordingly, the spacing and the angle between the filter units 120, 130 may allow for better flow through each filter, improving the yield.

In some embodiments, the filter units 120, 130 are conical (or otherwise have a triangular cross section) where the front seam distance and the back seam distance are equal. A conical or triangular filter may rise towards a peak portion towards the middle of the inlet chamber 310, and fall towards the container walls 200 to define the first trough 342. Alternatively, the conical filter may define the first trough 342 towards the middle of the inlet chamber 310, and rise towards the container walls 200 to define the first peak 352. A similar configuration may apply with respect to the intermediate chamber 330 and the outlet chamber 320 when defining the second peak 362 and the second trough 372.

To reduce the likelihood of the filter units 120, 130 touching and blocking each other, the filter units 120, 130 may not be nested; for example, the first filter unit 120 does not extend into or towards the second filter unit 130, or vice versa.

### System and Kit

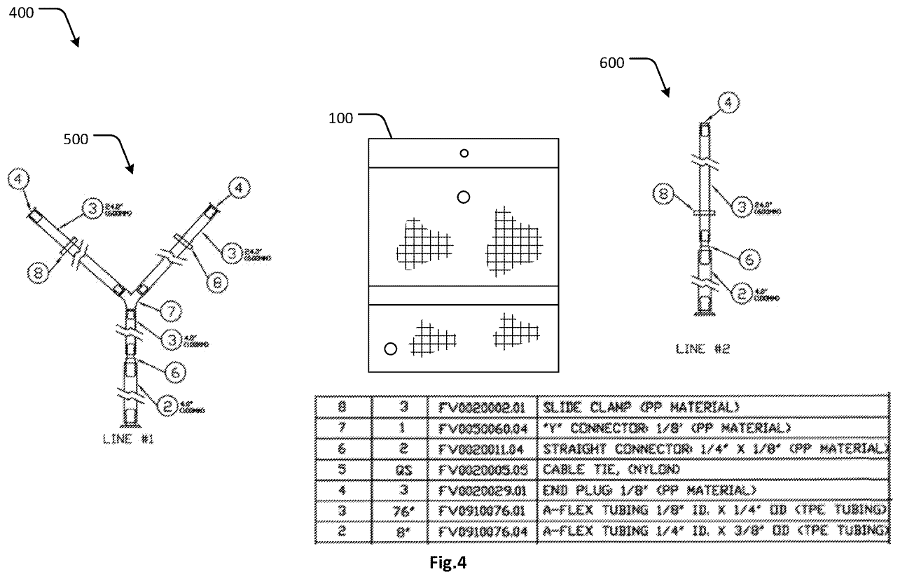

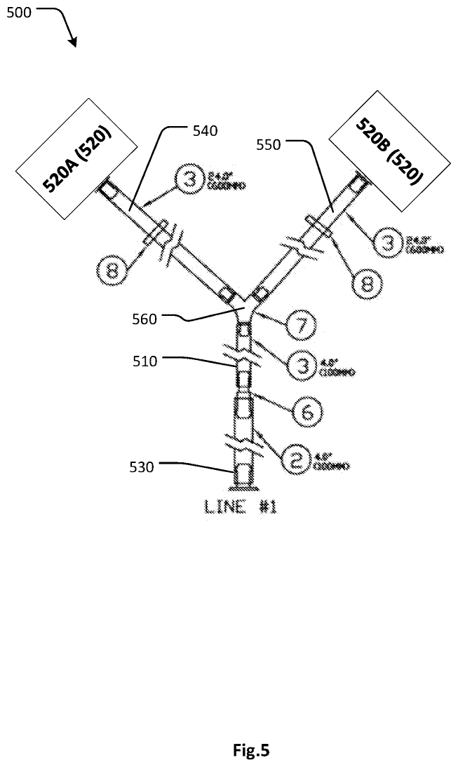

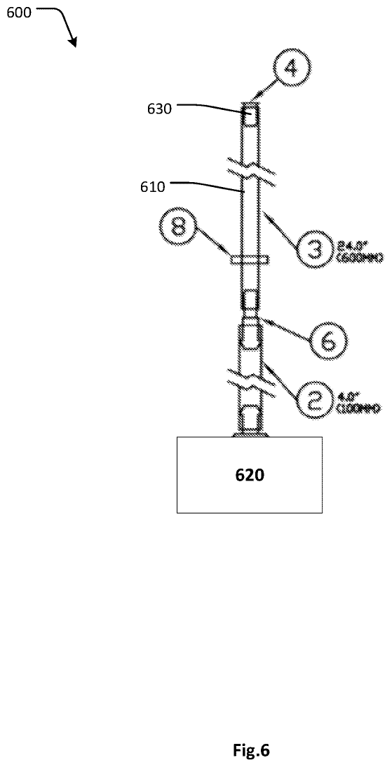

FIG. 4 shows a system 400 for filtering cells. The system may be supplied as a kit, wherein the system 400 is at least partly disassembled. The kit comprises the filtration device 100 of FIGS. 1-3, an inlet conduit 500 (FIG. 5), and an outlet conduit 600 (FIG. 6).

The inlet conduit 500 comprises a filling tube 510 adapted for coupling to the inlet port 108 of the filtration device 100, so as to allow fluid communication with the inlet chamber 310. In use, the filling tube 510 may receive a fluid from a fluid source 520, and direct it into the inlet chamber 310. The filling tube 510 may be connected to the inlet port 108 via an inlet coupling 530. The filling tube 510 may be in fluid communication with an inlet valve, which can be used to allow, prevent, and/or control flow of the fluid into the inlet chamber 310. In some embodiments, the inlet coupling 530 comprises the inlet valve.

The outlet conduit 600 comprises a drain tube 610 adapted for coupling to the outlet port 110 of the filtration device 100, so as to allow fluid communication with the outlet chamber 320. In use, the drain tube 610 may receive the yield (filtered fluid), which may be the fluid with the first and second matter removed, and direct it elsewhere, such as to one or more vials 620. The drain tube 610 may be connected to the outlet port 110 via an outlet coupling 630. The drain tube 610 may be in fluid communication with an outlet valve, which can be used to allow, prevent, and/or control flow of the filtered fluid from the outlet chamber 320. In some embodiments, the outlet coupling 630 comprises the outlet valve.

Some uses of the system 400 may involve receiving fluid from a plurality of sources. In such a scenario, the fluid may be the same fluid; for example, to ensure a continuous supply of the fluid through the filtration device 100, two of the fluid sources 520 may be connected to the system 400 so that when the first fluid source 520A runs empty (e.g. a tank), the second fluid source 520B can supply the fluid with minimal/zero interruption. In another scenario, the system 400 may receive two (or more) different types of fluid. This may allow two fluids to be mixed at or immediately prior to filtration. For example, when a cell composition is being formulated for filtration, the first fluid source 520A may contain the cells in a formulation buffer (e.g. 2X formulation buffer), and the second fluid source 520B may contain a cryoprotectant for protecting the filtered cells from damage during the process of cryopreservation. In an example, the cryoprotectant may be DMSO, which shows some cytotoxicity. Accordingly, the cryoprotectant may be added immediately prior to filtration to minimise cell exposure to DMSO, and allow more time for filling the filtered cell fluid into vials for freezing.

Accordingly, in some embodiments, the inlet conduit 500 may further comprise a first inlet tube 540 and a second inlet tube 550. The first inlet tube 540 is adapted to receive a first fluid from a first fluid source 520A, and the second inlet tube 550 is adapted to receive a second fluid from a second fluid source 520B, said first and second fluids being different from each other. In some embodiments, the first inlet tube 540 may be in fluid communication with a first supply valve, and the second inlet tube 550 may be in fluid communication with a second supply valve. The first and second supply valves can be used to allow, prevent, and/or control flow of the fluid from the fluid sources 520A, 520B.

The inlet tubes 540, 550 are configured to be in fluid communication with the filling tube 510. In some embodiments, the inlet conduit 500 comprises a manifold 560. The filling tube 510 is configured to be in fluid communication with the manifold 560 and adapted to receive fluid from at least one of the first and second inlet tubes 540, 550. The manifold 560 may be a Y-shaped or T-shaped connector, through which the first and second fluids can flow from the inlet tubes 540, 550 and into the filling tube 510. Accordingly, the inlet tubes 540, 550 and the filling tube 510 may form a Y-shape or a T-shape.

The various tubes 510, 540, 550, 610 may have an inner diameter in the range of ⅛ inches to ¾ inches. The various tubes 510, 540, 550, 610 may have an outer diameter in the range of ¼ inches to 1 inch. For example, in some embodiments, at least one of the tubes 510, 540, 550, 610 has an inner diameter of ⅛ inches and an outer diameter of ¼ inches. However, other size tubing may be used depending on the flow properties of the fluid (e.g. viscosity) and/or the desired throughput (flow rates, yield quality) of the system 400.

The kit may have one or more of its constituent parts (the filtration device 100, the inlet and outlet conduits 500, 600) made of DMSO compatible plastic. In some embodiments, all of the kit's constituent parts are made of DMSO compatible plastic. For example, the DMSO compatible plastic may be a plastic comprising one or more of polyethylene terephthalate (PET), High Density Polyethylene (HDPE), Low Density Polyethylene (LDPE), Polytetrafluoroethylene (PTFE), Polypropylene (PP), Poly Vinyl Chloride (PVC), and Thermo Plastic Elastomer (TPE).

The kit may have one or more of its constituent parts may comprise sterile welded parts, such as to connect the filter units 120, 130 to the container 102. The system 400, kit, and/or filtration device 100 may adhere to USP 788.

The kit may further comprise a pump which creates pressure in the system 400 to draw the fluid through the filtration device 100. The pump may be connected to the outlet conduit 600, and be used to assist with priming the system 400 (removal of air bubbles). When the fluid begins to flow into the container 102, the pump may be operated to suck air out of the container 102, creating a pressure differential that encourages the fluid to flow through the filter units 120, 130 and the chambers 300. The removal of air may be preferred over the introduction of air (via the inlet conduit 500), as introducing air to the system 400 may cause air bubbles to appear in the filtered fluid and reduce the amount/volume of filtered fluid yielded.

### Operation

In some embodiments, fluid may flow through the filtration device 100 by gravity, without mechanical assistance. In use, the container 102 is placed in an upright position wherein the inlet port 108 is oriented higher than the outlet port 110. In some embodiments, the container 102 comprises the flange 112 at the inlet end 104. The flange 112 may define the aperture 114 or connector through which the container 102 can be suspended from a hook to maintain the upright position.

In the upright position, the first filter unit 120 forms the tilted base of the inlet chamber 310 (tilted lid of the intermediate chamber 330), and the second filter unit 130 forms the tilted base of the intermediate chamber 330 (tilted lid of the outlet chamber 320). In the upright position, fluid flow through the filter units 120, 130 is assisted by gravity.

Prior to use, the system 400 should be “primed”. Priming involves removing air bubbles in the system 400 (such as in the inlet and outlet conduits 500, 600). To remove air bubbles, the inlet and outlet conduits 500, 600 may be mechanically manipulated by hand. The container 102 may also be selectively squeezed to push air out of the container 102, forcing the air bubbles along the inlet and outlet conduits 500, 600. During operation, the filtration device 100 may require intermittent priming.

In some embodiments, the filtration device 100 may be connected to the pump which creates pressure in the system 400 to draw the fluid through the filtration device 100. The pump may be particularly helpful in scenarios where the filtration device 100 is to be used in a configuration where gravity is insufficient or adversely affecting flow of the fluid from the inlet port 108 to the outlet port 110. For example, the pump may assist fluid flow where the inlet port 108 and the outlet port 110 are on/close to the same level. The pump may be connected to the outlet conduit 600. The pump may assist with priming.

When fluid flows into the inlet chamber 310, it contacts the tilted/tilted base of the inlet chamber 310 (the first filter unit 120). The first filter unit 120 stops the first matter from flowing through, and the first matter may be washed down into the first trough 342. The fluid passing through the first filter unit 120 may be referred to as a first filtered fluid.

The first filtered fluid passes through the first filter unit 120 into the intermediate chamber 330. The first filtered fluid contacts the tilted base of the intermediate chamber 330 (the second filter unit 130) and is filtered through the second filter unit 130. The second filter unit 130 is, in some embodiments, tilted at a different angle to the first filter unit 120. The second matter is stopped from flowing through the second filter unit 130, and the second matter may be washed down into the second trough 372. The fluid passing through the second filter unit 130 may be referred to as a second filtered fluid.

The second filtered fluid passes through the second filter unit 130 into the outlet chamber 320. The second filtered fluid collects in the outlet chamber 320 and is drained out via the outlet port 110.

The filtration device 100 and kit containing same may be used to filter a variety of fluids, such as stem cell culture medium, cell compositions, and harvested cells suspended in cell culture medium or another appropriate buffer.

Some embodiments of the present disclosure relate to a method of filtering a stem cell culture medium. The method comprises using the filtration device 100. The method may further comprise using the kit, wherein the stem cell culture medium is fed through the inlet conduit 500, through the filtration device 100 and out via the outlet conduit 600. The outlet conduit 600 may convey the filtered stem cell culture medium to be dispensed into at least one vial 620.

Some embodiments of the present disclosure relate to a method of filtering harvested cells in suspension. The harvested cells may be in the presence of enzymes for detachment or formulation reagents for filling. The method comprises using the filtration device 100. The method may further comprise using the kit. The outlet conduit 600 may convey the filtered harvested cells to be dispensed into at least one vial 620.

Some embodiments of the present disclosure relate to a method of purifying a cell composition. The method comprises passing cultured cells through a dual screen mesh filter such as the filtration device 100, thereby reducing visible particulates and/or cell aggregates. The kit may be used to perform the method. The outlet conduit 600 may convey the purified cell composition to be dispensed into at least one vial 620.

The first filter unit 120 of the filtration device 100 may have an average pore size of between 130 μm and 170 μm, such as 150 μm. The second filter unit 130 of the filtration device 100 may have an average pore size of between 20 μm and 60 μm, such as 40 μm. The purified cell composition may be substantially free of visible particles. The inspection process may be performed by trained and qualified operators through the naked eye, such as per USP 790 and USP 1790. A light box with a black and white background may be used to perform the inspection. No magnification or polarising of light may need to be used for this inspection.

The cultured cells may be provided in serum free cell culture medium. In some embodiments, the cultured cells are provided in cell culture medium which comprises serum such as fetal bovine serum (FBS). As would be known to those familiar with cell culture, cell culture medium comprises various components to support cell growth and these will largely be dependent on the cells in culture. Exemplary cell culture medium and components in the same are discussed below.

In some embodiments of the method, the cells are culture expanded.

In some embodiments of the method, the cells are mesenchymal lineage precursor or stem cells (MLPSCs).

After passing the cells through the filtration device 100, recovery of viable cell concentration may be between 60% and 100%. For example, 100% recovery of the viable cells means that the yield/filtered fluid contains all of the cells contained in the fluid introduced into the inlet chamber 310. A 60% recovery would indicate that of the cells contained in the fluid introduced into the inlet chamber 310, 40% of these cells remained in the filtration device 100; for example, trapped in the first filter unit 120 along with the first matter or trapped in the second filter unit 130 along with the second matter. In some embodiments, the recovery is between 70% and 90%.

In some embodiments of the method, the purified cell composition exhibits a D90 of less than 150 μm, preferably less than 100 μm, more preferably less than 50 μm.

### Chamber Volume and Relationship to Filter Unit Surface Area

The relationship between chamber volume and filter unit surface area should be considered when sizing the chambers 300 and filter units 120, 130. If the filter unit has too small a surface area in relation to the volume of the chamber which feeds the filter unit, the filter unit is likely to act as a bottleneck or choke point. This may slow down the overall filtration process, affecting the yield. The bottleneck may also result in the filter unit becoming clogged.

In some embodiments, the filter unit is tilted inside the filter container 102. By tilting the filter unit relative to the walls 200 of the container 102, a filter unit with a larger surface area can be fitted inside the container 102 compared to a configuration where the filter unit is not tilted.

If the filter unit is not tilted inside the container 102, the container 102 may have to be increased in size accommodate the filter unit. Increasing the size of the container 102 may affect the chamber volume, which as described above must be proportionate to the filter surface area in order to reduce the likelihood of the filter unit becoming a bottleneck or choke point. Increasing the size of the container 102 may be difficult if the container 102 is to be used in small or awkwardly-shaped spaces.

The relative volumes of the inlet chamber 310, intermediate chamber 330, and outlet chamber 320 should also be considered in order to promote fluid flow. It is usually not adverse to fluid flow if the subsequent chamber (e.g. the intermediate chamber 330) has a larger volume than the volume of the preceding chamber (e.g. the inlet chamber 310, which feeds the intermediate chamber 330). However, if the subsequent chamber volume is too small in comparison to the preceding chamber volume, fluid may enter the subsequent chamber volume faster than it leaves. This may result in a bottleneck or choke point. The filter unit pore size and fluid flow rates via the inlet and outlet ports 108, 110 also affect the speed of fluid movement through the various chambers 300 in the filter, and consequently influence the sizing of chamber volumes.

In some embodiments, all of the inlet chamber 310, the intermediate chamber 330, and the outlet chamber 320 have volumes that are approximately equal. The chamber volumes may accordingly be expressed in the ratio of 1:1:1.

In some embodiments, two of the inlet chamber 310, the intermediate chamber 330, and the outlet chamber 320 have volumes that are approximately equal. The other chamber may have a volume that is smaller than the volume of at least one of the other chambers. For example, the other chamber may have a volume that is between 30% to 70% of the volume of one of the other chambers.

In some embodiments, the intermediate chamber 330 is double the size of the inlet and outlet chambers 310, 320. The chamber volumes may accordingly be expressed in the ratio of 1:2:1 (inlet:intermediate:outlet).

In some embodiments, the intermediate chamber 330 and the outlet chamber 320 have volumes that are approximately equal. The inlet chamber 310 may have a smaller volume. The chamber volumes may be expressed in the ratio of 1:2:2 (inlet:intermediate:outlet).

In some embodiments, the inlet chamber 310 and the intermediate chamber 330 have volumes that are approximately equal. The outlet chamber 320 may have a smaller volume. The chamber volumes may be expressed in the ratio of 2:2:1 (inlet:intermediate:outlet).

In some embodiments, the inlet chamber 310, the intermediate chamber 330, and the outlet chamber 320 have volumes that are different to each other. For example, the volume of the intermediate chamber 330 may be three times larger than the inlet chamber 310, and the volume of the outlet chamber 320 may be three times larger than the inlet chamber 310. The chamber volumes may accordingly be expressed in the ratio of 1:3:2.

In some embodiments, the intermediate chamber 330 has the largest volume of all the chambers. By making the intermediate chamber 330 the most voluminous chamber, the fluid flowing through the first filter unit 120 can accumulate in the intermediate chamber 330 before passing through the second filter unit 130, thereby encouraging smooth fluid flow through the filtration device 100. In some embodiments, the container 102 has an overall volume of 1.5 litres, and which may allow approximately 2 litres of fluid to pass through with minimal fouling or clogging of the filter units 120, 130. In some embodiments, the container 102 has an overall volume of 2.0 litres.

In embodiments where the outlet chamber 320 is smaller than the intermediate chamber 330, the rate of fluid flow draining via the outlet port 110 can be controlled so that excess fluid does not accumulate in the outlet chamber 320. This reduces the risk of excess fluid in the outlet chamber 320 being adverse to fluid flow from the intermediate chamber 330.

### Methods

In an embodiment, the present disclosure relates to a method of filtering a cell culture medium comprising cells, the method comprising passing the cells through a filter described herein. For example, the cells can be passed through a screen mesh filter as discussed above. Cell type is not particularly limited when using the methods of the present disclosure which, in certain examples, are relevant to both differentiated and undifferentiated cells. In an example, the cells are mesenchymal lineage or precursor cells (MLPSCs). In this example, the methods of the present disclosure can be used to filter stem cell culture medium. For example, MLPSCs can be cultured according to the methods discussed below and then filtered by passing the cells through a filter described herein. In an example, the cells are provided in a stem cell culture medium. In an example, the stem cell culture medium is purified or partially purified before the resulting composition comprising the cells is passed through a filter described herein. For example, stem cell culture medium comprising cells may be subjected to centrifugation to remove culture medium from the cells. Cells may then be resuspended in an appropriate buffer or resuspension medium. In this example, the cells may be subject to multiple rounds of centrifugation and resuspension in order to wash the cells. Centrifuged cells are then resuspended in an appropriate buffer or resuspension medium and passed through a filter disclosed herein.

In certain embodiments, cells passed through a filter disclosed herein may be referred to as a purified cell composition. In an example, the purified cell composition is characterised by certain structural features specifically identifiable via visual inspection or other analytical method. For example, the purified cell composition may be substantially free of visible particles. In another example, the purified cell composition is characterised by size of aggregates. As used herein, “aggregate” means the total of a plurality of individual cells together in a cluster grouped by one or more adhesive properties including aggregation, agglomeration and agglutination. As used herein, “aggregation” means the tendency for cells to aggregate. In an example, 90% of the cell population/aggregate (e.g. stem cell population/aggregate) diameter (D90) is less than 150 μm. For example, a purified stem cell composition encompassed by the present disclosure can have a D₉₀ of less than 150 μm. In another example, the D₉₀ is less than 100 μm. In another example, the D₉₀ is less than 50 μm. For example, the D₉₀ can be between 50 μm and 150 μm. In another example, the D₉₀ can be between 50 μm and 100 μm.

In certain embodiments, passing cells or cell culture medium or resuspension medium comprising the same through a filter described herein provides a viable cell concentration between 60% and 100%. In some embodiments, the recovery of viable cells is between 70% and 90%. In an example, percentage recovery of viable cells is determined relative to the number of viable cells in the composition prior to it being passed through a filter described herein. In another example, the percentage is determined relative to the total number of cells in a purified cell composition (i.e. after filtration). Various routine methods of determining cell viability are known in the art. In an example, automated cell counting is used. Such methods may use techniques and apparatus based on coulter counting or flow cytometry. These systems generally rely on counting the number of specific size particles through electrical or optical detection and, in some instances may also incorporate dyes (or similar) to distinguish dead cells from live cells. In another example, semi-automated or manual cell counting may be used to determine cell viability. Such methods generally involve labelling dead cells using assays involving well known substances such as trypan blue or propidium iodine (PI) and then counting the number of dead cells relative to live cells in a sample. As will be appreciated by those of skill in the art, cell viability is generally assessed in samples that are representative of a larger composition with results from the representative sample being extrapolated to provide an overall level of cell viability for the larger composition.

### Mesenchymal Precursor Lineage or Stem Cells

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny.

Mesenchymal lineage precursor or stem cells can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal lineage precursor or stem cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells. In this regard, the term “population of cells enriched for mesenchymal lineage precursor or stem cells” will be taken to provide explicit support for the term “population of cells comprising X % mesenchymal lineage precursor or stem cells”, wherein X % is a percentage as recited herein. The mesenchymal lineage precursor or stem cells can, in some examples, form clonogenic colonies, e.g. CFU-F (fibroblasts) or a subset thereof (e.g., 50% or 60% or 70% or 70% or 90% or 95%) can have this activity.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the mesenchymal lineage precursor or stem cells are CD29+, CD54+, CD73+, CD90+, CD102+, CD105+, CD106+, CD166+, MHC1+MSCs.

In an example, the mesenchymal lineage precursor or stem cells are culture expanded from a population of MSCs that express markers, including CD73, CD90, CD105 and CD166, and lack expression of hematopoietic cell surface antigens such as CD45 and CD31. For example, the mesenchymal lineage precursor or stem cells can be culture expanded from a population of MSCs that are CD73+, CD90+, CD105+, CD166+, CD45− and CD31−. In an example, the population of MSCs is further characterized by low levels of major histocompatibility complex (MHC) class I. In another example, the MSCs are negative for major histocompatibility complex class II molecules, and are negative for costimulatory molecules CD40, CD80, and CD86. In an example, the culture expansion comprises 5 passages.

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture.

In one example, isolated or enriched mesenchymal lineage precursor or stem cells are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% O₂.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+ (TNAP+).

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+ (HSP-90β), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, mesenchymal lineage precursor or stem cells are obtained from a single donor, or multiple donors where the donor samples or mesenchymal lineage precursor or stem cells are subsequently pooled and then culture expanded.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

### Culture Expansion of Cells

In an example, mesenchymal lineage precursor or stem cells are culture expanded. “Culture expanded” mesenchymal lineage precursor or stem cells media are distinguished from freshly isolated cells in that they have been cultured in cell culture medium and passaged (i.e. sub-cultured). In an example, culture expanded mesenchymal lineage precursor or stem cells are culture expanded for about 4-10 passages. In an example, mesenchymal lineage precursor or stem cells are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 10 passages. In another example, mesenchymal lineage precursor or stem cells can be culture expanded for more than 7 passages. In these examples, stem cells may be culture expanded before being cryopreserved to provide an intermediate cryopreserved MLPSC population. In an example, compositions of the disclosure are prepared from an intermediate cryopreserved MLPSC population. For example, an intermediate cryopreserved MLPSC population can be further culture expanded prior to administration. Accordingly, in an example, mesenchymal lineage precursor or stem cells are culture expanded and cryopreserved. In an embodiment of these examples, mesenchymal lineage precursor or stem cells can be obtained from a single donor, or multiple donors where the donor samples or mesenchymal lineage precursor or stem cells are subsequently pooled and then culture expanded. In an example, the culture expansion process comprises:
i. expanding by passage expansion the number of viable cells to provide a preparation of at least about 1 billion of the viable cells, wherein the passage expansion comprises establishing a primary culture of isolated mesenchymal lineage precursor or stem cells and then serially establishing a first non-primary (P1) culture of isolated mesenchymal lineage precursor or stem cells from the previous culture;
ii. expanding by passage expansion the P1 culture of isolated mesenchymal lineage precursor or stem cells to a second non-primary (P2) culture of mesenchymal lineage precursor or stem cells; and,
iii. preparing and cryopreserving an in-process intermediate mesenchymal lineage precursor or stem cells preparation obtained from the P2 culture of mesenchymal lineage precursor or stem cells; and,
iv. thawing the cryopreserved in-process intermediate mesenchymal lineage precursor or stem cells preparation and expanding by passage expansion the in-process intermediate mesenchymal lineage precursor or stem cells preparation.

In an example, the expanded mesenchymal lineage precursor or stem cell preparation has an antigen profile and an activity profile comprising:
i. less than about 0.75% CD45+ cells;
ii. at least about 95% CD105+ cells;
iii. at least about 95% CD166+ cells.

In an example, the expanded mesenchymal lineage precursor or stem cell preparation is capable of inhibiting IL2Ra expression by CD3/CD28-activated PBMCs by at least about 30% relative to a control.

In an example, culture expanded mesenchymal lineage precursor or stem cells are culture expanded for about 4-10 passages, wherein the mesenchymal lineage precursor or stem cells have been cryopreserved after at least 2 or 3 passages before being further culture expanded. In an example, mesenchymal lineage precursor or stem cells are culture expanded for at least 1, at least 2, at least 3, at least 4, at least 5 passages, cryopreserved and then further culture expanded for at least 1, at least 2, at least 3, at least 4, at least 5 passages before being administered or further cryopreserved.

In an example, the majority of mesenchymal lineage precursor or stem cells in compositions of the disclosure are of about the same generation number (i.e., they are within about 1 or about 2 or about 3 or about 4 cell doublings of each other). In an example, the average number of cell doublings in the present compositions is about 20 to about 25 doublings. In an example, the average number of cell doublings in the present compositions is about 9 to about 13 (e.g., about 11 or about 11.2) doublings arising from the primary culture, plus about 1, about 2, about 3, or about 4 doublings per passage (for example, about 2.5 doublings per passage). Exemplary average cell doublings in present compositions are any of about 13.5, about 16, about 18.5, about 21, about 23.5, about 26, about 28.5, about 31, about 33.5, and about 36 when produced by about 1, about 2, about 3, about 4, about 5, about 6, about 7, about 8, about 9, and about 10 passages, respectively.

The process of mesenchymal lineage precursor or stem cell isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although mesenchymal lineage precursor or stem cells can generally withstand a certain amount of insult during preparation, cells are preferably manipulated by handling procedures and/or equipment that adequately performs the given step(s) while minimizing insult to the cells.

In an example, mesenchymal lineage precursor or stem cells are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a mesenchymal lineage precursor or stem cell composition according to the present disclosure is 95% homogeneous with respect to being CD105 positive and CD166 positive and being CD45 negative. In an example, this homogeneity persists through ex vivo expansion; i.e. though multiple population doublings. In an example, the composition comprises at least one therapeutic dose of mesenchymal lineage precursor or stem cells and the mesenchymal lineage precursor or stem cells comprise less than about 1.25% CD45+ cells, at least about 95% CD105+ cells, and at least about 95% CD166+ cells. In an example, this homogeneity persists after cryogenic storage and thawing, where the cells also generally have a viability of about 70% or more.

### Cell Culture Medium

Mesenchymal lineage precursor or stem cells disclosed herein can be culture expanded in various suitable culture mediums. The term “medium” or “media” as used in the context of the present disclosure, includes the components of the environment surrounding the cells. The media contributes to and/or provides the conditions suitable to allow cells to grow. Media may be solid, liquid, gaseous or a mixture of phases and materials. Media can include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to.

The cell culture media used for culture expansion contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest. For example, it may be necessary to increase the level of glucose (or other energy source) in the basal medium, or to add glucose (or other energy source) during the course of culture, if this energy source is found to be depleted and to thus limit growth. In an example, dissolved oxygen (DO) levels can also be controlled.

In an example, the cell culture medium contains human derived additives. For example, human serum and human platelet cell lysate can be added to the cell culture media. For example, the medium can comprise between 2 and 10% human serum. In an example, the medium comprises 3% v/v human serum.

In an example, the cell culture medium contains only human derived additives. Thus, in an example, the cell culture media is xeno-free. For avoidance of doubt, in these examples, the culture medium is free of animal proteins.

In an example, the culture medium comprises serum. In other examples the culture medium is fetal bovine serum free culture medium comprising growth factors that promote mesenchymal lineage precursor or stem cell proliferation. In an embodiment, the culture medium is serum free stem cell culture medium. In an example, the cell culture medium comprises:
a basal medium;
platelet derived growth factor (PDGF);
fibroblast growth factor 2 (FGF2).

In an example, the culture medium comprises platelet derived growth factor (PDGF) and fibroblast growth factor 2 (FGF2), wherein the level of FGF2 is less than 5 ng/ml. For example, the FGF2 level may be between 1 and 2 ng/ml.

In an example, the PDGF is PDGF-BB. In an example, the level of PDGF-BB is between about 1 ng/ml and 150 ng/ml. In another example, the level of PDGF-BB is between about 7.5 ng/ml and 20 ng/ml. In another example, the level of PDGF-BB is at least 10 ng/ml.

In other examples, additional factors can be added to the cell culture medium. In an example, the culture medium further comprising EGF. EGF is a growth factor that stimulates cell proliferation by binding to its receptor EGFR. In an example, the level of EGF is between 0.1 and 7 ng/ml. For example, the level of EGF can be at least 5 ng/ml. In an example, the level of EGF is between about 2 ng/ml and 5 ng/ml.

In the above examples, basal medium such as Alpha MEM or StemSpan™ can be supplemented with the referenced quantity of growth factor.

In other examples, additional factors can be added to the cell culture medium. For example, the cell culture media can be supplemented with one or more stimulatory factors selected from the group consisting of epidermal growth factor (EGF), 1α,25-dihydroxyvitamin D3 (1,25D), tumor necrosis factor α (TNF-α), interleukin-1β (IL-1β) and stromal derived factor 1α (SDF-1α).

In another example, the cell culture medium promotes stem cell proliferation while maintaining stem cells in an undifferentiated state. Stem cells are considered to be undifferentiated when they have not committed to a specific differentiation lineage. As discussed above, stem cells display morphological characteristics that distinguish them from differentiated cells. Furthermore, undifferentiated stem cells express genes that may be used as markers to detect differentiation status. The polypeptide products may also be used as markers to detect differentiation status. Accordingly, one of skill in the art could readily determine whether the methods of the present disclosure maintain stem cells in an undifferentiated state using routine morphological, genetic and/or proteomic analysis.

### Prototype and Experimental Data

The inventors tested filtration devices 100 of the configuration shown in FIGS. 1-3, wherein the container 102 had an overall volume in the range of 1.5 litres to 2 litres. The first filter unit 120 had a pore size of 150 μm and a surface area of approximately 258 cm2. The second filter unit 130 had a pore size of 40 μm and a surface area of 180 cm2. The chamber volumes are summarised in Table 1 below.

TABLE 1

| | | Inlet | Intermediate | Outlet |
|---|---|---|---|---|
| | | chamber | chamber | chamber |
| Total volume | Chamber | volume | volume | volume |
| of chamber | ratio | (cm2) | (cm2) | (cm2) |
| 1.5 litres (1500 cm2) | 1:1:1 | 500 | 500 | 500 |
| 2.0 litres (2000 cm2) | | 670 | 670 | 670 |
| 1.5 litres (1500 cm2) | 1:2:1 | 375 | 750 | 375 |
| 2.0 litres (2000 cm2) | | 500 | 1000 | 500 |
| 1.5 litres (1500 cm2) | 1:2:2 | 300 | 600 | 600 |
| 2.0 litres (2000 cm2) | | 400 | 800 | 800 |
| 1.5 litres (1500 cm2) | 2:2:1 | 600 | 600 | 300 |
| 2.0 litres (2000 cm2) | | 800 | 800 | 400 |
| 1.5 litres (1500 cm2) | 1:3:2 | 250 | 750 | 500 |
| 2.0 litres (2000 cm2) | | 330 | 1000 | 670 |

In view of the minimum and maximum chamber volumes in Table 1, the chamber volumes to filter unit surface area ratio are therefore:

Inlet chamber:
Minimum inlet chamber volume=250 cm3
Maximum inlet chamber volume=800 cm3
Accordingly, the inlet chamber volume is in the range of approximately 0.9 (250/258) to approximately 3.2 (800/258) times the surface area of the first filter unit.

### Intermediate Chamber:

Minimum intermediate chamber volume=500 cm3
Maximum intermediate chamber volume=1000 cm3
Accordingly, the intermediate chamber volume is in the range of approximately 1.9 (500/258) to approximately 4.0 (1000/258) times the surface area of the first filter unit.
Minimum intermediate chamber volume=500 cm3
Maximum intermediate chamber volume=1000 cm3
Accordingly, the intermediate chamber volume is in the range of approximately 2.7 (500/180) to approximately 5.6 (1000/180) times the surface area of the second filter unit.

### Outlet Chamber:

Minimum outlet chamber volume=300 cm3
Maximum outlet chamber volume=800 cm3
Accordingly, the outlet chamber volume is in the range of approximately 1.6 (300/180) to approximately 4.5 (800/180) times the surface area of the second filter unit.

The filter units 120, 130 were tilted relative to each other and connected to the container walls 200, to form a “V” configuration when viewed in cross section (such as shown in FIGS. 2A-2D and FIGS. 3A and 3B). The distance between the front wall seams 340, 360 (where the first and second filter units 120, 130 were respectively coupled to the front wall 202 of the container 102) was approximately 1 inch.

To determine filtration efficiency of the filtration device 100, the inventors used beads to represent particles of >40 μm size and other beads to represent cells. The beads representing particles of >40 μm were Cospheric Red coloured beads Cat #UVPMS-BR-1.20 45-53 μm (1.20 g/cc density and 45-53 μm size range). The beads used to represent cells were Cospheric Blue coloured beads Cat #BLPMS-1.08 20-27 μm (1.08 g/cc density and 20-27 μm size range). Both sets of beads were mixed in a matrix (MES Stop from Lonza Singapore) having the same density as a matrix containing 10% DMSO (BloodStor 100) in αMEM cell culture medium.

The filtration device 100 was also tested using only the Cospheric Red coloured beads 45-53 μm representing particles of >40 μm size in the BloodStor 100 matrix.

Some embodiments of the filtration device 100 were tested using cells in the BloodStor 100 matrix representing a quenched cell suspension (1 L trypLE+4 L of v2.2 medium).

Some embodiments of the filtration device 100 were tested using cells concentrated by centrifugation at 400×g for 8 minutes at 2-8° C. The cells were resuspended in 400 mL modified αMEM (SGTS-10533, Lonza) to represent a concentrated cell suspension.

Some embodiments of the filtration device 100 were tested using cells concentrated by centrifugation at 400×g for 8 minutes at 2-8° C. The cells were resuspended in 300 mL cryomedium containing Plasmalyte A, 5% Human serum albumin and 10% DMSO to represent a 1× Drug Product.

To determine the amount of cells filtered from the fluid (comprising the beads in the matrix), two 1 mL samples were taken both before and after filtration. The number of both types of beads in a 10 μL aliquot were counted using a glass hemocytometer, or using the trypan blue exclusion method. The amount of beads measured pre-filtration and post-filtration were compared to determine the filtration efficiency. The weight of the fluid was measured before and after filtration to determine the amount of fluid retained in the filtration device 100.

The inventors found that the filtration device 100 as shown in FIGS. 1-3 successfully filtered out beads representing particles of >40 μm size without impeding the flow through of beads representing cells (20-27 μm). Though a few red coloured beads (representing particles of >40 μm) were noted in the post-filter sample, these were similar in size to the 20-27 μm beads. Therefore, the count for 45-53 μm beads was considered “zero”. The particulate vs cell removal performance did not appear to be affected by the various matrices tested, notably even those containing 10% DMSO.

To provide a comparison of performance, the inventors also tested a commercially available filtration device (Haemonetics SQ40S) having a 40 μm filter unit/screen. This filtration device successfully filtered out beads representing particles of >40 μm size. However, the flow through of beads representing cells (20-27 μm) was impeded, resulting in a 21.9% loss of the smaller beads.

The inventors observed that in some embodiments, some of the 20-27 μm beads (representing cells) remained attached to the inside of the chambers 300. The matrix (in which the beads/cells were contained) flowed through the filter units 120, 130 without much resistance. The inventors observed that the amount of residual beads could be reduced by allowing the filtration device 100 to accumulate about ⅓ or ½ of its volume capacity (˜300-500 mL). To enable the filtration device 100 to accumulate in this manner, a clamp or valve was used to temporarily stop the flow of fluid out of the filtration device 100 (such as a clamp or valve in fluid communication with the drain tube 610). Once the filtration device 100 was about ⅓ full to ½ full, the outlet clamp/valve was removed. In some embodiments, this was observed to allow better flow of the fluid through the filtration device 100. The inventors observed in some embodiments that the accumulated fluid reduced the likelihood of the walls 200 of the filtration device 100 touching and acting as a bottleneck, thereby inducing residual beads build-up. The accumulated fluid was observed in some embodiments to allow improved control of the purification process from a handling perspective.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A filter comprising:
a flexible container defining an inlet port toward an inlet end of the container and an outlet port toward an outlet end of the container, the container having one or more walls connecting the inlet and outlet ends; and
a first filter unit and a second filter unit spaced apart within the container, wherein:
the first filter unit is coupled to the container to define an inlet chamber in fluid communication with the inlet port;
the second filter unit is coupled to the container to define an outlet chamber in fluid communication with the outlet port; and
the first and second filter units define an intermediate chamber between the inlet and outlet chambers, the first filter unit being configured to filter a fluid flowing from the inlet chamber to the intermediate chamber, and the second filter unit being configured to filter the fluid flowing from the intermediate chamber to the outlet chamber;
wherein the inlet chamber comprises a first trough relative to a first peak;
wherein the intermediate chamber comprises a second trough relative to a second peak; and
wherein the outlet port and the second filter unit are configured to be spaced apart when the fluid is flowing through the second filter unit into the outlet chamber.

2. The filter of claim 1, wherein the outlet port and the second filter unit are configured to be spaced apart by a spacer system.

3. A filter comprising:
a container defining an inlet port toward an inlet end of the container and an outlet port toward an outlet end of the container, the container having flexible front and back walls connecting the inlet and outlet ends; and
a first filter unit and a second filter unit spaced apart within the container, wherein:
the first filter unit is coupled to the front and back walls of the container to define an inlet chamber in fluid communication with the inlet port;
the second filter unit is coupled to the front and back walls of the container to define an outlet chamber in fluid communication with the outlet port; and
the first and second filter units define an intermediate chamber between the inlet and outlet chambers, the first filter unit being configured to filter fluid flowing from the inlet chamber to the intermediate chamber, and the second filter unit being configured to filter fluid flowing from the intermediate chamber to the outlet chamber;
wherein the first filter unit is coupled to the front wall along a first front seam, and the second filter unit is coupled to the front wall along a second front seam;
wherein the first filter unit is coupled to the back wall at a first back seam, and the second filter unit is coupled to the back wall at a second back seam;
wherein a distance between the first and second front seams is less than a distance between the first and second back seams; and
wherein the outlet port and the second filter unit are configured to be spaced apart when the fluid is flowing through the second filter unit into the outlet chamber.

4. The filter of claim 3, further comprising a spacer system configured to space apart the outlet port and the second filter unit, and at least one of:
(i) the front wall and the first filter unit;
(ii) the back wall and the first filter unit; and
(iii) the back wall and the second filter unit.

5. The filter of claim 3 or claim 4, wherein the first filter unit is connected to the front wall of the container at an acute angle to define an inlet chamber trough.

6. The filter of claim 5, wherein the first filter unit is connected to the back wall of the container at an obtuse angle.

7. The filter of any one of claims 3 to 6, wherein the second filter unit is connected to the back wall of the container at an acute angle to define an intermediate chamber trough.

8. The filter of claim 7, wherein the second filter unit is connected to the front wall of the container at an acute angle.

9. The filter of any one of claims 1 to 8, wherein the first filter unit comprises a mesh defining pores, the pores having an average pore size of 130 μm to 170 μm.

10. The filter of claim 9, wherein the first filter unit pores have an average pore size of 150 μm.

11. The filter of any one of claims 1 to 10, wherein the second filter unit comprises a mesh defining pores, the pores having an average pore size of 20 μm to 60 μm.

12. The filter of claim 11, wherein the second filter unit pores have an average pore size of 40 μm.

13. The filter of any one of claims 1 to 12, wherein the first filter unit is substantially flat.

14. The filter of any one of claims 1 to 13, wherein the second filter unit is substantially flat.

15. The filter of claim 4, or any one of claims 5 to 14 when dependent on claim 3, wherein the spacer system comprises a separator.

16. The filter of claim 4, or any one of claims 5 to 14 when dependent on claim 3, wherein the spacer system comprises a clip.

17. The filter of claim 4, or any one of claims 5 to 14 when dependent on claim 3, wherein the spacer system comprises a magnet system.

18. The filter of claim 4, or any one of claims 5 to 14 when dependent on claim 3, wherein the spacer system comprises a brace.

19. The filter of any one of claims 3 to 18, wherein the second back seam angles the second filter unit and the outlet port away from each other.

20. The filter of any one of claims 3 to 18, wherein the front wall is configured to bulge away from the second filter unit.

21. A filter comprising:
a flexible bag defining an inlet port toward an inlet end of the bag and an outlet port toward an outlet end of the bag, the bag having opposed front and back walls connecting the inlet and outlet ends;
a first filter unit and a second filter unit spaced apart within the bag, wherein:
the first filter unit is coupled to the bag to define an inlet chamber in fluid communication with the inlet port;
the second filter unit is coupled to the bag to define an outlet chamber in fluid communication with the outlet port;
the first and second filter units define an intermediate chamber between the inlet and outlet chambers, the first filter unit being configured to filter fluid flowing from the inlet chamber to the intermediate chamber, and the second filter unit being configured to filter fluid flowing from the intermediate chamber to the outlet chamber; and
wherein the outlet port and the second filter unit are configured to be spaced apart when the fluid is flowing through the second filter unit into the outlet chamber.

22. The filter of claim 21, wherein the first filter unit and the second filter unit are not parallel.

23. A kit for filtering cells, the kit comprising:
the filter of any one of claims 1 to 22;
an inlet conduit, the inlet conduit comprising:
a first inlet tube and a second inlet tube, the first and second inlet tubes fluidly connected to a manifold;
a filling tube, the filling tube in fluid communication with the manifold and adapted to receive fluid from at least one of the first and second inlet tubes; and
an inlet coupling adapted to connect the filling tube to the inlet port of the filter and allow fluid communication with the inlet chamber;
an outlet conduit comprising:
a drain tube; and
an outlet coupling adapted to connect the drain tube to the outlet port of the filter and allow fluid communication with the outlet chamber and the drain tube.

24. The kit of claim 23, wherein the inlet tubes and the filling tube form a Y-shape or a T-shape.

25. The kit of claim 23 or claim 24, wherein the inlet tubes have an inner diameter of ⅛ inches and an outlet diameter of ¼ inches.

26. The kit of any one of claims 23 to 25, wherein: (i) the filling tube has an inner diameter of ⅛ inches and an outlet diameter of ¼ inches; or (ii) the drain tube has an inner diameter of ⅛ inches and an outlet diameter of ¼ inches.

27. The kit of any one of claims 23 to 26, further comprising an outlet valve or clamp configured to control fluid flow through the drain tube.

28. The filter or kit of any one of claims 1 to 27, wherein one or more or all of the container, the bag, the filter units 120, 130, or the tubes are made of DMSO compatible plastic, preferably a plastic comprising one or more of polyethylene terephthalate (PET), High Density Polyethylene (HDPE), Low Density Polyethylene (LDPE), Polytetrafluoroethylene (PTFE), Poly Vinyl Chloride (PVC), Thermo Plastic Elastomer (TPE), and Polypropylene (PP).

29. The filter or kit of any one of claims 1 to 28, when used for filtering stem cell culture medium.

30. A method of filtering a stem cell culture medium, the method comprising using the filter of any one of claims 1 to 22 or the kit of any one of claims 23 to 28.

31. A method of purifying a cell composition, comprising passing cultured cells through a dual screen mesh filter, thereby reducing visible particulates and/or cell aggregates, wherein the dual screen mesh filter comprises a first filter screen with an average pore size of between 130 μm and 170 μm and a second filter screen with an average pore size of between 20 μm and 60 μm.

32. The method of claim 31, wherein the cultured cells are provided in serum free cell culture medium.

33. The kit of any one of claims 23 to 28 or method of claim 31 or claim 32, wherein the cells are culture expanded.

34. The kit or method of claim 33, wherein the cells are mesenchymal lineage precursor or stem cells (MLPSC)s.

35. The method of any one of claims 31 to 34, wherein after passing the cells through the dual screen mesh filter, recovery of viable cell concentration is between (i) 60% and 100%; or (ii) 70% and 90%.

36. The method of any one of claims 31 to 35, wherein the purified cell composition exhibits a D90 of less than 150 μm, preferably less than 100 μm, more preferably less than 50 μm.

37. The method of any one of claims 31 to 36, wherein the purified cell composition is substantially free of visible particles.

38. A filter comprising:
a flexible container defining an inlet port toward an inlet end of the container and an outlet port toward an outlet end of the container, the container having one or more walls connecting the inlet and outlet ends;
a first filter unit comprising a first filter mesh; and
a second filter unit comprising a second filter mesh;
wherein the first filter unit and the second filter unit are spaced apart within the container;
wherein the first filter unit is coupled to the container to define an inlet chamber in fluid communication with the inlet port;
wherein the second filter unit is coupled to the container to define an outlet chamber in fluid communication with the outlet port; and
wherein the first and second filter units define an intermediate chamber between the inlet and outlet chambers, the first filter unit being configured to filter a fluid flowing from the inlet chamber to the intermediate chamber, and the second filter unit being configured to filter the fluid flowing from the intermediate chamber to the outlet chamber;
wherein the inlet chamber comprises a first trough relative to a first peak;
wherein the intermediate chamber comprises a second trough relative to a second peak; and
wherein at least one of the first filter mesh and the second filter mesh is configured to be spaced apart from the one or more walls when the fluid is flowing through the filter.

39. A filter comprising:
a container defining an inlet port toward an inlet end of the container and an outlet port toward an outlet end of the container, the container having flexible front and back walls connecting the inlet and outlet ends;
a first filter unit comprising a first filter mesh; and
a second filter unit comprising a second filter mesh;
wherein the first filter unit and the second filter unit are spaced apart within the container, wherein:
the first filter unit is coupled to the front and back walls of the container to define an inlet chamber in fluid communication with the inlet port;
the second filter unit is coupled to the front and back walls of the container to define an outlet chamber in fluid communication with the outlet port; and
the first and second filter units define an intermediate chamber between the inlet and outlet chambers, the first filter unit being configured to filter fluid flowing from the inlet chamber to the intermediate chamber, and the second filter unit being configured to filter fluid flowing from the intermediate chamber to the outlet chamber;
wherein the first filter unit is coupled to the front wall along a first front seam, and the second filter unit is coupled to the front wall along a second front seam;
wherein the first filter unit is coupled to the back wall at a first back seam, and the second filter unit is coupled to the back wall at a second back seam;
wherein a distance between the first and second front seams is less than a distance between the first and second back seams;
wherein the outlet port and the second filter unit are configured to be spaced apart when the fluid is flowing through the second filter unit into the outlet chamber; and
wherein at least one of the first filter mesh and the second filter mesh is configured to be spaced apart from the front or back walls when the fluid is flowing through the filter.

40. The steps, features, integers, compositions and/or compounds disclosed herein or indicated in the specification of this application individually or collectively, and any and all combinations of two or more of said steps or features.
